(19) United States
(12) Patent Application Publication
(10) Pub. No.: US 2024/0408081 A1
(43) Pub. Date: Dec. 12, 2024

(54) NOVEL SYNERGISTIC COMBINATIONS AND METHODS OF USE THEREOF FOR TREATING CANCERS

(71) Applicants: UNIVERSITE DE BORDEAUX, Bordeaux (FR); INSTITUT NATIONAL DE LA SANTE ET DE LA RECHERCHE MEDICALE, Paris (FR)

(72) Inventors: Christophe GROSSET, Pessac (FR); Martin HAGEDORN, Pessac (FR); Farah RAHAL, Paris (FR); Fatma Zohra KHOUBAI, Paris (FR)

(21) Appl. No.: 18/691,570

(22) PCT Filed: Sep. 15, 2022
(86) PCT No.: PCT/EP2022/075708

§ 371 Date: Mar. 13, 2024

(30) Foreign Application Priority Data
Sep. 17, 2021 (EP) 21197506.5

(51) Int. Cl. A61K 31/496; A61K 31/282; A61K 31/366; A61K 31/40; A61K 31/5377; A61K 33/243; A61P 35/00

(52) U.S. Cl. CPC A61K 31/496; A61K 31/282; A61K 31/366; A61K 31/40; A61K 31/5377; A61K 33/243; A61P 35/00

(57) ABSTRACT

The invention relates to novel compositions, combinations and methods relating to compounds which inhibit EZH2 and their uses for treating and/or preventing tumors associated with methyltransferase EZH2. More specifically the invention relates to synergistic bi-therapy compositions for use in a method of treating and/or preventing tumors associated with methyltransferase EZH2, comprising administering to a subject in need, a therapeutically effective amount of the composition, wherein said composition comprises the combination of EZH2 inhibitor and one HMG-COA reductase inhibitor or statin. The present invention also relates to synergistic tri-therapy compositions as well as methods of use thereof for treating and/or preventing specific types of cancers and tumors comprising further administering an effective amount of one or more anti cancer drugs or chemotherapeutic agents in combination with the above bi-therapy compositions.

### FIELD OF INVENTION

The invention relates to novel synergistic combinations as well as methods of use for treating and/or preventing specific types of cancers and tumors, such as in particular cancers and tumors associated with histone methyltransferase Enhancer of Zeste Homolog 2 (EZH2). The present invention also relates to pharmaceutical compositions comprising said synergistic combinations for use in the treatment and/or the prevention of histone methyltransferase EZH2-associated cancers and/or tumors. The present invention further relates to a new marker, the Enhancer of Zeste Homolog 2 (EZH2), as well as use of EZH2 as prognosis factor of higher relapse or death probabilities and to a method of analyzing the biomarker EZH2 so as to predict the prognosis of a subject suffering from specific types of cancers.

### BACKGROUND OF THE INVENTION

Epigenetic modification could regulate chromatin state and gene expression through DNA methylation and demethylation, histone modification, chromatin remodeling etc. . . . , without altering DNA sequences. Polycomb group proteins (PcGs), a group of important epigenetic regulators, play an important part in cell proliferation and are critical factors of pluripotency and differentiation of stem cells as well as aberrant gene expression during the malignant transformation. PcG proteins contain two core complexes, respectively are the maintenance complex polycomb repressive complex 1 (PRC1) and the initiation complex polycomb repressive complex 2 (PRC2). PRC1 has been known to mono-ubiquitinate the histone H2A at Lys 119 through RING1A and RING1B ubiquitin ligases. PRC2 has been considered to catalyze the mono-, di-, and tri-methylation of histone H3 at Lys 27 to regulate gene transcription.

Enhancer of zeste homolog 2 (EZH2) is an evolutionary conserved gene identified in many species, sharing similar structural motifs and domains. The histone methyltransferase EZH2 has been identified as a catalytic subunit of PRC2 for tri-methylation of histone H3 at Lys 27 (H3K27me3) by SET domain in its C-terminus, which silences targeted genes and is involved in various biological functions (e.g., cell cycle, cell proliferation, cell differentiation, etc. . . . ). The role of EZH2 in cancer progression has also been considered. EZH2 is aberrantly overexpressed in various malignant tumors, such as prostate cancer, breast cancer, and ovarian cancer. EZH2 mediates H3K27me3 and functions as a critical factor in promoting tumor growth and metastasis in many malignant tumor models. In addition, EZH2 gain, or loss of function mutations have also been discovered in various cancer. Some studies indicate that inhibition of EZH2 by small molecular inhibitors or gene knockdown results in reduced cancer cell growth and tumor formation. Beyond playing its role in a PCR2-dependent manner as a histone modifier, EZH2 also acts in a PCR2 and histone independent manner in cancer. For example, EZH2 can methylate non-histone protein STAT3 in glioblastoma, and participate in androgen receptor-associated complexes in castration-resistant prostate cancer (CRPC) as a co-activator. As described by Gan L et al., (Biomark Res 6, 10 (2018). doi.org 10.1186 s40364-018-0122-2), the diverse functions of EZH2 in cancer derive from its genetic, transcriptional, post-transcriptional and post-translational regulation in different circumstances and different types of cancer.

Further findings to support an oncogenic role for EZH2 have more recently emerged. Studies have shown that recurrent heterozygous point mutations at tyrosine 641 (Y641) within the C-terminal catalytic SET domain of EZH2 occur in 22% of germinal center B-cell (GCB) diffuse large cell B-cell lymphomas (DLBCL) and in 7% to 12% of follicular lymphomas (FL). This was shown to confer gain-of-function of enzyme activity resulting in augmented conversion of H3K27 to the trimethylated form. Y641 mutants (Y641F, Y641N, Y641S, Y641C, and Y641H) have reduced methylation activity of unmethylated H3K27 but enhanced activity for the dimethylated version of H3k27. The mutant thus cooperated together with wild-type EZH2 to shift the steady state of H3K27 to favor trimethylation and thus repressed expression of Polycomb targets. EZH2 point mutations at the A677 and A687 residues have also been identified in non-Hodgkin lymphomas (NHL), where they similarly result in hypertrimethylation of H3K27. Additional support for a gain-of-function role for mutant EZH2 in cancer came from the identification of cancer-associated loss-of-function mutations in other chromatin regulators that normally antagonized EZH2 activity. UTX (ubiquitously transcribed tetratricopeptide repeat gene on X chromosome) is a histone demethylase that functions in part by antagonizing EZH2 activity by removing methyl groups from di- and trimethylated H3K27. Inactivating mutations affecting UTX occur in several types of human cancer, including multiple myeloma, medulloblastoma, esophageal cancer, bladder cancer, pancreatic cancer, and renal cancers. These mutations include homozygous (in females) or hemizygous (in males) large deletions, nonsense mutations, small frame-shifting insertion/deletions, and consensus splice site mutations that lead to a premature termination codon. Almost all mutations are predicted to result in loss of the JmjC domain of UTX, which is essential for its demethylase activity, and have been shown to cause increased levels of H3K27 trimethylation. Therefore, as described by Kim K H et al. (Targeting EZH2 in cancer. Nat Med. 2016; 22 (2): 128-134. doi: 10.1038 nm.4036), loss-of-function mutations in UTX may be analogous to those caused by gain-of-function mutations in EZH2.

In spite of this understanding, the efficacy of EZH2 inhibitors for preventing and/or treating tumors and cancer has remained controversial. Several candidates have been or are currently in clinical trials and the data available thus far is not conclusive.

The first EZH2 inhibitor was 3-deazaneplanocin A (DZNep). DZNep, a known S-adenosyl-L-homocysteine (SAH) hydrolase inhibitor, indirectly inhibits EZH2 through the increase of SAH, which directly represses S-adenosyl-L-methionine-dependent histone methyltransferase activity. DZNep globally inhibits histone methylation but is not specific to EZH2.

A second candidate GSK-126—also known as GSK2816126—was shown to inhibit wild type and Y641 mutant EZH2 with similar potency and is highly selective compared to EZH1 (150-fold increased potency) or 20 other methyltransferases (>1000-fold selective for EZH2). A multicentric phase 1 clinical trial was initiated back in 2019 to evaluate the safety, maximum-tolerated dose (MTD), pharmacokinetics, and pharmacodynamics of GSK-126 (NCT02082977) in patients with relapsed/refractory solid tumors and hematologic malignancies. Forty-one participants (21 solid tumors, 20 lymphomas) received escalating doses of GSK2816126 ranged from 50 to 3000 mg twice weekly as an intravenous solution for 28 days (3 weeks on/1 week off). This clinical phase was however terminated since the results showed insufficient evidence of clinical activity and did not justify further clinical investigation while the dosing method and relatively short half-life limited effective exposure (See clinical trial No. NCT02082977). The results of this clinical phase were also reported by Yap T A et al. (Clin Cancer Res. 2019 Dec. 15; 25 (24): 7331-7339) concluded that GSK2816126 was not a viable drug to target EZH2 in patients with refractory/relapsed solid and hematologic malignancies, despite preclinical data showing sensitivity of multiple solid tumor and lymphoma cell lines. The study defined the maximum tolerated dose (MTD) of GSK2816126 as 2,400 mg, but at this level, the drug showed inadequate clinical activity, with off-target dose-limiting toxicity (DLT) precluding further dose escalation.

Several other S-adenosyl-methionine-competitive inhibitors of EZH2 were also developed including inter alia GSK343, GSK926 and tazemetostat (E7438/EPZ6438). GSK926 and GSK343 can suppress histone H3K27me3 level and inhibit EZH2 activity in breast and prostate cancer cells, while GSK343 can only be used in vitro due to the high clearance in rat pharmacokinetic studies.

Tazemetostat showed improved potency and pharmacokinetic properties and can be administered orally. Wiese M et al. (Klin Padiatr. 2016 April; 228 (3): 113-7) investigated the correlation of expression of EZH2 and other PRC2 genes (EZH1, SUZ12, EED) with overall survival of pediatric glioblastoma multiform (GBM) patients and the cytotoxic impact of EZH2 inhibition by tazemetostat in pediatric glioblastoma multiform/diffuse intrinsic pontine glioma/diffuse midline glioma (GBM/DIPG/DMG) cells harboring either a H3.3 mutation or a H3 wildtype. Wiese et al. however concluded that the treatment with this EZH2 inhibitor does not represent a promising approach in these tumors. Still the USFDA granted on June 2020 an accelerated approval to the EZH2 inhibitor tazemetostat (TAZVERIK, Epizyme, Inc.) for adult patients with relapsed or refractory (R/R) follicular lymphoma (FL) whose tumors are positive for an EZH2 mutation as detected by an FDA-approved test and who have received at least 2 prior systemic therapies, as well as for adult patients with R/R FL who have no satisfactory alternative treatment options.

As such, there is currently a need for new methods and compositions with greater therapeutic efficacy for treating and/or preventing the numerous malignancies associated with methyltransferase EZH2 activity. The present invention addresses this need and provides novel therapies.

### SUMMARY OF THE INVENTION

The present invention relates to synergistic bi-therapy compositions as well as methods of use thereof for treating and/or preventing specific types of cancers and tumors comprising administering to a subject in need a therapeutically effective amount of the composition, wherein said composition comprises the combination of EZH2 inhibitors and one statin.

The present invention also relates to synergistic tri-therapy compositions as well as methods of use thereof for treating and/or preventing specific types of cancers and tumors comprising further administering an effective amount of one or more anticancer drugs or chemotherapeutic agents in combination with the above bi-therapy compositions.

Synergistic bi-therapy and tri-therapy compositions according to the present invention are particularly effective in treating specific types of cancer and tumors associated with histone methyltransferase EZH2.

Most importantly, synergistic bi-therapies and tri-therapies according to the present invention allow reducing by at least half the dose regimen of EZH2 inhibitors compared to previous clinical trials, and thus exhibit a much-reduced toxicity while retaining therapeutic efficiencies against said tumors. Indeed, Applicants showed herein below that administering the combinations of one EZH2 inhibitor such as GSK-126 with at least one statin and/or one or more anticancer drugs or chemotherapeutic agents sensitize the cancers or tumors to GSK-126, thereby reversing or reducing the resistance of patients to GSK-126.

The present invention further provides to the use of EZH2 as a biomarker of poor prognosis in some cancer subjects, particularly for patients suffering from hepatoblastoma or Diffuse Intrinsic Pontine Glioma (DIPG) and to a method of predicting a poor prognosis of said subjects comprising determining levels of EZH2 in isolated samples of said subjects.

### BRIEF DESCRIPTION OF THE FIGURES

FIGS. 1A-E: show that DMG cells are sensitive to GSK-126 inhibitor at low doses and that GSK-126 blocks H3k27me3 trimethylation. (A) EZH2 transcripts are significantly overexpressed in a series of DMG biopsies (n=35) compared to normal brains (n=10), microarray data retrieved from GSE50021. Dashed square delimits samples regrouped around median expression. Parametric t test between DMG biopsies versus normal brains. (B) Patient-derived cell lines express EZH2 protein detected by Western Blot. (C-D) GSK-126 strongly inhibits proliferation of NEM157i and SU-DIPG-IVi DMG cells (C) and induces tumor cell death after 5 days of treatment (D). One way ANOVA (n>3, p<0.0001), Bonferroni's multiple comparisons post-test. (E) H3k27me3 trimethylation is reduced in a dose-dependent manner in GSK-126 treated NEM157i and SU-DIPG-IVi DMG cells. One blot representative of 3 independent experiments. *, p<0.05; **, p<0.01; ***, p<0.001.

FIGS. 2A-C: show that DMG cells are sensitive to GSK-126 inhibitor at low doses. (A-C) the cell viability of SU-DIPG-IVi (A), NEM157i (B), and NEM163i (C) DMG cells was measured after 72 h of treatment in presence of increasing doses of GSK-126 (n=3). For each cell line, IC50 is shown in the corresponding graph.

FIG. 3: shows the increased expression of genes involved in lipid and cholesterol synthesis in GSK-126-treated DIPG cells. Graphs show the normalized levels of HMGCS, HMGCR, LDLR, NPC1 and SQLE transcripts measured by real-time quantitative PCR in NEM163i, NEM157i, and SU-DIPG-IVi cells treated by 1.5% DMSO or GSK-126 (IC₅₀ measured in each cell line, n=6, Unpaired Mann & Whitney test). GAPDH transcript was used as internal control for normalization. ** p<0.01; *** p<0.001.

FIG. 4: shows the increased expression of SQLE and HMGCR proteins in GSK-126-treated DIPG cells. Levels of HMGCR and SQLE proteins measured by Western in NEM163i and SU-DIPG-IVi cells treated by 1.5% DMSO or GSK-126 (IC₅₀ measured in each cell line, n=4 to 5, Unpaired Mann & Whitney test). GAPDH protein was used as internal control for normalization. Representative blots of 2 to 4 independent experiments are shown *p<0.05; ** p<0.01.

FIGS. 5A-C: show the absence of effects of inhibitors of cholesterol biosynthesis pathway enzymes (doses shown on X-axis) on the viability of SU-DIPG-IVi and NEM157i DMG cells. (A-C) Three different chemical inhibitors of different enzymes implicated in the cholesterol biosynthesis pathway and induced by GSK-126 [(A) Terbinafine: squalene epoxidase—SQLE, (B) Atorvastatin: hydroxymethylglutaryl-coenzyme A (HMG-CoA) reductase—HMGSC1, (C) ACSS2 inhibitor: acetate-dependent acetyl-CoA synthetase 2—ACCS2] have no effect on DMG cell proliferation up to 30 μM, suggesting insensibility of the cells to these inhibitors in the absence of GSK-126. One way ANOVA (n=3, p<0.0001), Bonferroni's multiple comparisons post-test. *, p<0.05; **, p<0.01.

FIGS. 6A-C: show synergistic effect of the bi-therapy comprising GSK-126 and inhibitors of cholesterol biosynthesis pathway enzymes on DMG cells. (A-C) Cell growth assays of NEM157i and SU-DIPG-IVi cells (as indicated) exposed to 4 μM of GSK-126, a dose which has no growth inhibitory effects on DMG cells (open circles), and to increasing doses of ACSS2 inhibitor (A), Atorvastatin (B) or Terbinafine (C). One way ANOVA (n=3, p<0.0001), Bonferroni's multiple comparisons post-test. Bi-therapy treatment shows significant proliferation inhibition starting at low micro molar doses of statins which show no effects alone (see FIG. 3). **, p<0.01; ***, p<0.001.

FIGS. 7A-E: show the molecular and phenotypical characteristics of the new primary DMG cell line BXdmg1. (A) BXdmg1 primary cell characterization. Upper left panel: bright field microscopy showing cell morphology of early passage BXdmg1 primary cells. Bar=200 μm. Upper middle panel: H&E staining of Cytoblock preparation of cells revealing nuclear irregularities. Upper right panel: Proliferation status using Ki-67 staining. Lower left panel: Demonstration of presence of H3K27M mutation in BXdmg1 cells. Lower middle panel: Status of H3K27me trimethylation in BXdmg1 cells. Lower right panel: Western blot on histone protein isolation demonstrating typical reduction of H3K27me after exposure to GSK-126. (B, C) BXdmg1 proliferation is inhibited by GSK-126 with an IC50 of 10.36 μM, a comparable sensitivity to the other DMG cells (see FIG. 2). One way ANOVA (n=3, p<0.0001), Bonferroni's multiple comparisons post-test. (D) BXdmg1 cells are not sensitive to exposure to cholesterol biosynthesis inhibitors. (E) Combo treatment of GSK-126 and Atorvastatin (Ator) shows stronger growth inhibition than GSK-126 or Atorvastatin alone. Combo effect is visible at low doses and is lost at higher doses of GSK-126 because of its cytotoxicity alone at these concentrations. One way ANOVA (n=3, p<0.0001), Bonferroni's multiple comparisons post-test. ns, non-significant; *, p<0.05; ** p<0.01; ***, p<0.001.

FIGS. 8A-C: show the synergistic effect of GSK-126 and inhibitors of cholesterol biosynthesis pathway enzymes on DMG cell migration. (A-C) Cell migration impairment in the combo treatment revealed by a wound scratch assay in the NEM157i (A) and NEM163i (B) cells and in the BXdmg1 primary cells (C). One way ANOVA (n=3, p<0.0001), Bonferroni's multiple comparisons post-test. ns, non-significant; *, p<0.05; **, p<0.01; ***, p<0.001.

FIGS. 9A-E: show the synergistic effect GSK-126 and inhibitors of cholesterol biosynthesis pathway enzymes on DMG spheroid formation. (A, B, D) Phase contrast micrographs of representative spheroids derived from indicated DMG glioma cells after exposure to indicated inhibitors. (C, E) Statistical analysis of treatment effects on the formation of spheroids derived from the DMG cell line shown above the corresponding graph. Phenotypic criterions were spheroid formed or not formed (dispersed cells). Fisher' Exact test was used to compare relevant treatments and the combination (combo). GSK-126 dose was 20 μM for NEM157i and 15 μM for the others. ***, p<0.001.

FIGS. 10A-C: show the synergistic effect GSK-126 and inhibitors of cholesterol biosynthesis pathway enzymes on DMG tumor development in mice. (A) Orthotopic tumor growth inhibition of SU-DIPG-IVi-Luc cells (expressing the Luciferase) implanted in the brainstem of NOD/LtSz-scid IL2R gamma (NSG) mice after GSK-126 intraperitoneal treatments. Tumor growth inhibition is demonstrated for all mice by bioluminescence live imaging and statistical analysis after the indicated treatments (solvent control: DMSO; GSK-126). Grey scale bar indicates level of expression, light grey values equal low and dark grey values high expression. Parametric/Student test (DMSO: n=18; GSK-126: n=23). (B) The same approach is performed at a non-cytotoxic dose of GSK-126 in combination with a non-cytotoxic dose of Atorvastatin. The combination of both drugs shows significant growth inhibitory effects, whereas single treatments are not effective. Bars indicate median plus range. One way ANOVA (DMSO: n=13; Atorvastatin: n=13; GSK-126: n=17; Combo: n=13; p<0.0001), Bonferroni's multiple comparisons post-test. (C) Similar tumor growth inhibitory results were obtained using a chick CAM DMG model where a less angiogenic phenotype of implanted tumors was observed in the combo treatment, whereas single treatments had no anti-angiogenic effect Two-sided Fisher's exact test. ns, non-significant; *, p<0.05; ** p<0.01.

FIGS. 11A-B: show that EZH2 silencing impedes hepatoblastoma development in vivo. (A-B) Huh6 cells (A) or HepG2 cells (B) were transfected with a control siRNA (SiCTR) or a siRNA against EZH2 (SiEZH2). 24 hours later, cells were collected and grafted on the chick chorioallantoic membrane (CAM) at day 10 of embryonic development. Tumor growth was monitored from day 11 to day 16. For both cell lines and the indicated condition, top left panels: representative pictures of tumors having grown on CAM (Lane 1) at day 17 and after resection and Paraformaldehyde fixation (lane 2). For both cell lines, top right panels: The number of CAMs presenting or not bleeding at Day 7 in SiCTR tumors versus SiEZH2 tumors is shown as bars. Two-sided Fisher's exact test. For both cell lines, bottom panels: On day 6 after SiEZH2 or SiCTR-transfected Huh6 cell implantation, tumors were resected and weighed. Left panels: representative pictures of extracted tumors in the indicated condition. Right panel: bars represent means+/−SD of tumor weight in mg. The total number of eggs analyzed per group is indicated in brackets above the corresponding condition. Parametric t Student test. **, p<0.01; ****, p<0.0001.

FIGS. 12A-B: show the effect of cisplatin alone or two EZH2 inhibitors on the survival of hepatoblastoma cells. (A-B) Huh6 and HepG2 cells were grown for 48 h in presence of increasing concentrations of cisplatin (A, top panels), GSK-126 (A, bottom panels) or EPZ-6438 (Tazemetostat, B) as indicated. Cell survival and IC50 were measured using the CellTiter 96® AQᵤₑₒᵤₛ One Solution Cell Proliferation Assay from Promega. IC50 for the corresponding condition is as shown.

FIGS. 13A-C: show the effect of combining cisplatin and GSK-126 on the viability of hepatoblastoma cells in 2D and 3D culture conditions. (A-B) Huh6 and HepG2 cells were grown for 72 h in classical 2D culture condition or as tumor spheroids in presence of GSK-126 alone, cisplatin alone, or a combination of both as indicated. The drug concentrations were selected to kill 50% of hepatoblastoma cells when used alone (see IC50 for each cell line and drug in FIG. 12). (A) Cell survival was measured using the CellTiter 96® AQᵤₑₒᵤₛ One Solution Cell Proliferation Assay from Promega (n=5, One-way ANOVA, p<0.0001), Sidak's multiple comparisons post-test. (B) Live cells were stained in green with Calcein-AM substrate (https://pubchem.ncbi.nlm.nih.gov/compound/Calcein-AM) and dead cells were stained in red using Ethidium homodimer (EthD-1, https://pubchem.ncbi.nlm.nih.gov/compound/12328897). Data show an additional antitumor effect of GSK-126 and cisplatin in vitro on hepatoblastoma cells growing in 2D and 3D conditions. ***, p<0.001; ****, p<0.0001.

FIGS. 14A-B: show that EZH2 depletion increases the sensitivity of hepatoblastoma cells to cisplatin. (A) Huh6 (on the left) and HepG2 (on the right) cells were transfected with Control (SiCTR) or two different SiEZH2 (SiEZH2-1 and SiEZH2-2) and grown for 72 h in presence of increasing concentrations of cisplatin. Cell survival was measured using the CellTiter 96® AQᵤₑₒᵤₛ One Solution Cell Proliferation Assay from Promega. (n=4, Two-way ANOVA, p<0.0001), Sidak's multiple comparisons post-test. Light grey asterisks: significant variations in cell viability between control cells and SiEZH2-1 transfected cells. Black asterisks: significant variations in cell viability between control cells and SiEZH2-2 transfected cells. (B) Control (SiCTR) or siEZH2-transfected HepG2 cells were grown for 48 h as tumor spheroids in presence of 12 μM of cisplatin (=IC50). Live cells were stained in green with Calcein-AM substrate and dead cells were stained in red using EthD-1 (see FIG. 11B). One representative illustration of 3 independent experiments. ns, non-significant; *, p<0.05; **, p<0.01.

FIGS. 15A-B: show that the statins synergize with GSK-126 to eradicate hepatoblastoma cells. (A) Huh6 cells (on the left) and HepG2 cells (on the right) were treated for 72 h in presence of increasing concentrations of Simvastatin or of Atorvastatin (n=3, One-way ANOVA, p<0.0001; Sidak's multiple comparisons post-test, *, p<0.05; **, p<0.01; ***, p<0.001; ****, p<0.0001. (B) Huh6 cells (on the left) and HepG2 cells (on the right) were treated for 72 h in presence of increasing concentration of GSK-126 (top panels) or of cisplatin (bottom panels) in absence of or in presence of 4 μM of Simvastatin or of 8 μM of Atorvastatin. (n=3, Two-way ANOVA, p<0.0001, Sidak's multiple comparisons post-test. (A-B) Cell viability and IC50 were measured in all conditions using the CellTiter 96® AQᵤₑₒᵤₛ One Solution Cell Proliferation Assay (Promega). Light grey asterisks: significant variations in cell viability between GSK-126 alone and GSK-126 combined with the indicated concentrations of Atorvastatin. Black asterisks: significant variations in cell viability between GSK-126 alone and GSK-126 combined with the indicated concentrations of Simvastatin. * p<0.05, ** p<0.01, *** p<0.001, **** p<0.0001.

FIGS. 16A-B: show that the statins synergize with GSK-126 in absence of or in presence of cisplatin to eradicate hepatoblastoma cells in vitro. (A-B) Huh6 cells (A) and HepG2 cells (B) were treated for 72 h by 3 μM of GSK-126 in combination or not with 3 μM of cisplatin in presence of increasing concentrations of Simvastatin (right panels) or of Atorvastatin (left panels). Cell viability was measured in each condition using the CellTiter 96x AQᵤₑₒᵤₛ One Solution Cell Proliferation Assay from Promega (n=3, Two-way ANOVA, p<0.0001; Sidak's multiple comparisons post-test. §: significant variations in cell viability between solvent control (DMSO) and GSK-126 alone or GSK-126 combined with any of the tested concentrations of the indicated statin. *: significant variations in cell viability between solvent control (DMSO) and GSK-126+cisplatin or GSK-126+cisplatin combined with any of the tested concentrations of the indicated statin. ^{#}: significant variations in cell viability between GSK-126 and GSK-126+cisplatin at the indicated concentration of the indicated statin. *, p<0.05; **, p<0.01; ****, p<0.0001; ^{##}, p<0.01; ^{###}, p<0.001; ^{####}, p<0.0001; ^{§§}, p<0.01; ^{§§§}, p<0.001; ^{§§§§}, p<0.0001;

FIG. 17: shows that statins strongly increase the sensitivity of hepatoblastoma cell to Tazemetostat (EPZ-6438). Huh6 cells (on the left) and HepG2 cells (on the right) were treated for 72 h in presence of increasing concentrations of Tazemetostat in absence of or in presence of 8 μM of Atorvastatin or of 4 μM of Simvastatin (as indicated in the legend below each graph). Cell viability and IC50 (see value in brackets for each condition) were measured for each condition using the CellTiter 96® AQᵤₑₒᵤₛ One Solution Cell Proliferation Assay from Promega (n=3, Two-way ANOVA, p<0.0001; Sidak's multiple comparisons post-test. Black asterisks: significant variations in cell viability between Tazemetostat alone and Tazemetostat in presence of Simvastatin. Light grey asterisks: significant variations in cell viability between Tazemetostat alone and Tazemetostat in presence of Atorvastatin. *, p<0.05; **, p<0.01; ***, p<0.001; ****, p<0.0001.

FIGS. 18a-c: show the increased expression of EZH2 in hepatoblastoma. In particular FIG. 18a shows the increased expression of EZH2 transcript in proliferative C2A subgroup compared to C1 and C2B subgroups, and non-tumoral samples (NT, in this figure and the following) from our dataset (gse104766, Hooks K B, Audoux J, Fazli H, Lesjean S, Ernault T, Dugot-Senant N, et al. New insights into diagnosis and therapeutic options for proliferative hepatoblastoma. Hepatology 2018; 68:89-10) (Wilcoxon matched pairs signed rank test). FIG. 18b is a graph recapitulating the Two-tailed Pearson R correlations between expression of EZH2 transcript and expression of TOP2A transcript (gse104766, Hooks K B, Audoux J, Fazli H, Lesjean S, Ernault T, Dugot-Senant N, et al. New insights into diagnosis and therapeutic options for proliferative hepatoblastoma. Hepatology 2018; 68:89-10). The R and p-value are as shown. FIG. 18c shows the increased expression of EZH2 transcript in hepatoblastomas and non-tumoral liver tissues from Ikeda's dataset (gse131329, Hiyama E. Gene expression profiling in hepatoblastoma cases of the Japanese Study Group for Pediatric Liver Tumors-2 (JPLT-2) trial: Science Repository OU; 2019 2019 Feb. 12), Carrillo-Reixach's dataset (gse133039, Carrillo-Reixach J, Torrens L, Simon-Coma M, Royo L, Domingo-Sabat M, Abril-Fornaguera J, et al. Epigenetic footprint enables molecular risk stratification of hepatoblastoma with clinical implications. J Hepatol 2020; 73:328-341), Lopez-Terrada's dataset (gse75271, Sumazin P, Chen Y, Trevino L R, Sarabia S F, Hampton O A, Patel K, et al. Genomic analysis of hepatoblastoma identifies distinct molecular and prognostic subgroups. Hepatology 2017; 65:104-121), Karns's dataset (gse81928, Valanejad L, Cast A, Wright M, Bissig K D, Karns R, Weirauch M T, et al. PARP1 activation increases expression of modified tumor suppressors and pathways underlying development of aggressive hepatoblastoma. Commun Biol 2018; 1:67), Buendia's dataset (Cairo S, Armengol C, De Reynies A, Wei Y, Thomas E, Renard C A, et al. Hepatic stem-like phenotype and interplay of Wnt/beta-catenin and Myc signaling in aggressive childhood liver cancer. Cancer Cell 2008; 14:471-484) and Kappler's dataset (gse151347, Eichenmuller M, Trippel F, Kreuder M, Beck A, Schwarzmayr T, Haberle B, et al. The genomic landscape of hepatoblastoma and their progenies with HCC-like features. J Hepatol 2014; 61:1312-1320). Unpaired Mann & Whitney test. * p<0.05; *** p<0.001; **** p<0.0001.

FIG. 19: shows a positive correlation between the levels of EZH2 and TOP2A transcripts in hepatoblastoma datasets. The graphs recapitulate the Two-tailed Pearson R correlations between expression of EZH2 transcript and expression of TOP2A transcript in HB samples from Ikeda's dataset (gse131329, Hiyama E. Gene expression profiling in hepatoblastoma cases of the Japanese Study Group for Pediatric Liver Tumors-2 (JPLT-2) trial: Science Repository OU; 2019 2019 Feb. 12), Carrillo-Reixach's dataset (gse133039, Carrillo-Reixach J, Torrens L, Simon-Coma M, Royo L, Domingo-Sabat M, Abril-Fornaguera J, et al. Epigenetic footprint enables molecular risk stratification of hepatoblastoma with clinical implications. J Hepatol 2020; 73:328-341), Lopez-Terrada's dataset (gse75271, Sumazin P, Chen Y, Trevino L R, Sarabia S F, Hampton O A, Patel K, et al. Genomic analysis of hepatoblastoma identifies distinct molecular and prognostic subgroups. Hepatology 2017; 65:104-121), Karns's dataset (gse81928, Valanejad L, Cast A, Wright M, Bissig K D, Karns R, Weirauch M T, et al. PARP1 activation increases expression of modified tumor suppressors and pathways underlying development of aggressive hepatoblastoma. Commun Biol 2018; 1:67), Buendia's dataset (Cairo S, Armengol C, De Reynies A, Wei Y, Thomas E, Renard C A, et al. Hepatic stem-like phenotype and interplay of Wnt/beta-catenin and Myc signaling in aggressive childhood liver cancer. Cancer Cell 2008; 14:471-484) and Kappler's dataset (gse151347, Eichenmuller M, Trippel F, Kreuder M, Beck A, Schwarzmayr T, Haberle B, et al. The genomic landscape of hepatoblastoma and their progenies with HCC-like features. J Hepatol 2014; 61:1312-1320). For each dataset, the R and p-value are as shown in the corresponding graph.

FIGS. 20a-e: show that EZH2 expression correlates with tumor recurrence and patient death. Correlative analyses between EZH2 transcript expression and clinical or histological features. FIG. 20a: stage (gse75271, Sumazin P, Chen Y, Trevino L R, Sarabia S F, Hampton O A, Patel K, et al. Genomic analysis of hepatoblastoma identifies distinct molecular and prognostic subgroups. Hepatology 2017; 65:104-121); FIG. 20b: histopathological subtypes as indicated (gse75271, Sumazin P, Chen Y, Trevino L R, Sarabia S F, Hampton O A, Patel K, et al. Genomic analysis of hepatoblastoma identifies distinct molecular and prognostic subgroups. Hepatology 2017; 65:104-121); FIG. 20c: remission and recurrence (gse133039, Carrillo-Reixach J, Torrens L, Simon-Coma M, Royo L, Domingo-Sabat M, Abril-Fornaguera J, et al. Epigenetic footprint enables molecular risk stratification of hepatoblastoma with clinical implications. J Hepatol 2020; 73:328-341; FIG. 20d: PRETEXT radiologic staging system (gse131329, Hiyama E. Gene expression profiling in hepatoblastoma cases of the Japanese Study Group for Pediatric Liver Tumors-2 (JPLT-2) trial: Science Repository OU; 2019 2019 Feb. 12); FIG. 20e: alive and dead patients (MAS5.0-u133a, Buendia et al dataset (Cairo S, Armengol C, De Reynies A, Wei Y, Thomas E, Renard C A, et al. Hepatic stem-like phenotype and interplay of Wnt/beta-catenin and Myc signaling in aggressive childhood liver cancer. Cancer Cell 2008; 14:471-484). One-way ANOVA, ** p<0.01, Sidak's multiple comparisons post-test for FIG. 20a-d. Unpaired Mann Whitney test for FIG. 20e. ns, not significant; * p<0.05; ** p<0.01; *** p<0.001; **** p<0.0001.

FIGS. 21a-e: show that the EZH2 silencing inhibits hepatoblastoma cell growth by inducing cell senescence. FIG. 21a: Level of EZH2 protein in control (siCTRL) and EZH2-depleted Huh6 (left) or HepG2 (right) cells using two different siRNAs (siEZH2-1 or siEZH2-2 as indicated in FIG. 21a-e and in the following figures). FIGS. 21a and d: Representative blots of 3 independent experiments or more are shown in cropped images (loading control: GAPDH). FIG. 21b: Growth (Absorbance at 565 nm) of EZH2-depleted Huh6 (left) or HepG2 (right) cells versus siCTRL cells (n=3, Two way-ANOVA, **** p<0.0001; Sidak's multiple comparisons post-test). FIG. 21c: Senescence measured in siCTRL versus EZH2-depleted Huh6 (left) or HepG2 (right) cells. Representative experiments are shown, and bar graphs recapitulate means±standard deviation (SD) (n=5, One way-ANOVA, *** p<0.001; Sidak's multiple comparisons post-test). FIG. 21d: Relative level of cell cycle inhibitor proteins P16 and P21 in EZH2-depleted Huh6 (left) or HepG2 (right) cells versus siCTRL. FIG. 21e: Graphs presenting caspase 3/7 activity in EZH2-depleted Huh6 (left) or HepG2 (right) cells versus siCTRL (n=3, One way-ANOVA, not significant; Sidak's multiple comparisons post-test). ns, not significant; ** p<0.01; *** p<0.001; **** p<0.0001.

FIGS. 22a-b: show that EZH2 silencing reduces the migration of hepatoblastoma cells and the trimethylation of histone H3 Lysine 27. FIG. 22a: Migration of EZH2-depleted Huh6 cells versus siCTRL using siEZH2-1 or siEZH2-2 as indicated. Top panel: representative images of 4 independent experiments. Bottom panel: bar graphs recapitulate means±SD (n=4, Two-way-ANOVA, **** p<0.0001; Sidak's multiple comparisons post-test). FIG. 22b: Relative levels of H3K27me3 and total histone H3 proteins in siCTRL versus EZH2-depleted Huh6 (left) or HepG2 (right) cells. Representative blots of 3 independent experiments are shown in cropped images (loading control: histone H3). *** p<0.001; **** p<0.0001.

FIGS. 23a-b: show that EZH2 potentiates ERK signaling in hepatoblastoma through its histone methyl transferase activity. FIG. 23a: shows the levels of total ERK and phospho-ERK (p-ERK) proteins in siCTRL versus EZH2-depleted Huh6 (left) or HepG2 (right) cells using siEZH2-1 or siEZH2-2 as indicated. FIG. 23b: shows the levels of total ERK and phospho-ERK (p-ERK) proteins in Huh6 (left) and HepG2 (right) cells expressing an empty gene cassette (LV-CTRL), a wild-type EZH2 protein (LV-EZH2 cassette) or the H698A mutated form of EZH2 protein (LV-EZH2* cassette). FIG. 23a-b: are representative blots of 3 experiments shown in cropped images (loading control: total proteins).

FIGS. 24a-e: show that EZH2 acts as an oncogene in hepatoblastoma through its methyl transferase activity. FIG. 24a: shows the coding sequence corresponding to EZH2 methyltransferase domain. Top panel: wild type (WT) sequence. Bottom panel: mutated sequence encoding an alanine (A) instead of histidine (H) on residue 689 (H698A mutant: referred to as EZH2*). FIG. 24b: shows the level of endogenous and transgenic EZH2 and EZH2* proteins in Huh6 (left) and HepG2 (right) cells transduced by LV-CTRL (empty gene cassette), LV-EZH2 or LV-EZH2*. Representative blots of 3 independent experiments or more are shown in cropped images (loading control: GAPDH). FIG. 24c: shows the cell growth (Absorbance at 565 nm) of transduced Huh6 (left) or HepG2 (right) cells expressing an empty cassette (LV-CTRL), the wild-type (LV-EZH2) or H698A mutated EZH2 protein (LV-EZH2*) (n=3, Two way-ANOVA, **** p<0.0001; Sidak's multiple comparisons post-test). FIG. 24d: shows the phase contrast micrographs of representative 96 hour-aged spheroids deriving from Huh6 (top) and HepG2 (bottom) cells expressing the CTRL cassette, EZH2 or EZH2*. FIG. 24e: shows graphs presenting spheroid surface area in mm² in Huh6 (left) and HepG2 (right) cells expressing an empty cassette (LV-CTRL), the wild-type (LV-EZH2) or H698A mutated EZH2 protein (LV-EZH2*) (n=4, One way-ANOVA, **** p<0.0001; Sidak's multiple comparisons post-test). ns. not significant: * p<0.05: ** p<0.01: *** p<0.001: **** p<0.0001.

FIG. 25: shows that cisplatin resistance is partly mediated by EZH2 methyl transferase activity in hepatoblastoma cells. Response to cisplatin of Huh6 (left) and HepG2 (right) cells expressing an empty cassette (LV-CTRL), the wild-type (LV-EZH2) or H698A mutated EZH2 protein (LV-EZH2*). Graphs show the percentage of viable Huh6 (left) and HepG2 (right) cells treated with increasing doses of cisplatin (n=3; bars=means+/−SD; Two way-ANOVA, **** p<0.0001; Sidak's multiple comparisons post-test). * p<0.05; *** p<0.001; **** p<0.0001.

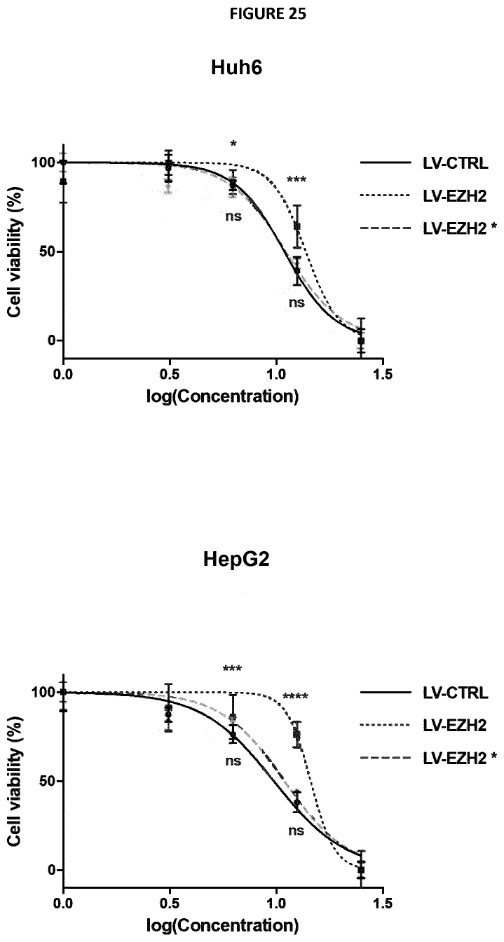

FIGS. 26a-b: show that EZH2 depletion decreases HMGCR level in hepatoblastoma cells. FIG. 26a: Volcano plot of protein level fold change (X-axis, log 2) versus q-values (Y-axis, log 10) measured by proteomic analysis in siEZH2-1 versus siCTRL Huh6 cells. Proteins significantly down- and up-regulated in EZH2-depleted cells are in the left and right dashed frames, respectively. HMGCR protein is depicted as a big black spot and shown by an arrow. FIG. 26b: Level of HMGCR protein in control (siCTRL) and EZH2-depleted Huh6 (left) or HepG2 (right) cells using two different siRNAs (siEZH2-1 or siEZH2-2 as indicated). Representative blots of 3 independent experiments or more are shown in image (loading control: GAPDH). The HMGCR protein is shown as a band of 98 kDa.

FIGS. 27a-b: show that the inactivation of EZH2 histone methyl transferase by GSK-126 induces HMGCR level and lipid synthesis in hepatoblastoma cells. FIG. 27a: Relative levels of H3K27me3 and total H3 proteins in control (DMSO-treated) versus GSK-126-treated Huh6 (left) or HepG2 (right) cells (GSK-126 used at IC₅₀; 6 and 8 μM for Huh6 and HepG2 cells, respectively). Representative blots of 3 independent experiments are shown in cropped images (loading control: histone H3). FIG. 27b: Level of HMGCR protein in control (DMSO) versus GSK-126-treated Huh6 (left) or HepG2 (right) cells. Representative blots of 3 independent experiments are shown in images (loading control: GAPDH). FIG. 27c: Level of lipids in control (DMSO) versus GSK-126-treated Huh6 (left) or HepG2 (right) cells. Lipid granules are shown as dark granules in the cytoplasm of cells. Representative images of 3 independent experiments are shown.

FIG. 28: shows that GSK-126 is less toxic than cisplatin in xenopus embryo. Xenopus embryos were grown in the presence of increasing concentrations of cisplatin or GSK-126 as indicated below the panels. Representative images of 3 independent experiments.

FIG. 29: shows that GSK-126 plus statin completely blocks the clonogenic capacity of hepatoblastoma cells. The survival and proliferation of hepatoblastoma cells was evaluated using a clonogenic assay. Huh6 (top panel) and HepG2 (bottom panel) cells were treated with DMSO (control: CTRL), GSK-126 at IC₂₅ dose (3 and 4 μM for Huh6 and HepG2 cells, respectively), statin (ATR: atorvastatin at 8 μM; SIM: simvastatin at 4 μM) or both. 10 days after cell plating, colonies were stained with crystal violet and plates were imaged using Fusion FX (Vilber Lourmat). Representative images of 3 independent experiments.

FIG. 30: shows that GSK-126 plus statin impedes the migration of hepatoblastoma cells. The migration of Huh6 cells was evaluated using a wound-healing assay. Huh6 cells were treated with DMSO (control: CTRL), GSK-126 at IC₂₅ dose (3 and 4 μM for Huh6 and HepG2 cells, respectively), statin (ATR: atorvastatin at 8 μM; SIM: simvastatin at 4 μM) or both. After cell plating and wound induction, the percentage of relative wound density was monitored using an Incucyte S3 Live-cell analysis instrument (Sartorius). Top panel: Representative images of 3 independent experiments of migrating Huh6 cells 20 hours after wound induction. Bottom panel: kinetic analysis of Huh6 cell migration treated by the indicated drugs. Bar graph recapitulates means±SD (n=3, Two-way-ANOVA at 24 hours' time point, **** p<0.0001; Sidak's multiple comparisons post-test). *** p<0.001.

FIGS. 31a-d: show that GSK-126-atorvastatin combination impedes hepatoblastoma development in vivo without affecting liver and renal functions. FIG. 31a: Schematic representative of the different groups of mice treated with the vehicle (3-times a week, PBS with 1% DMSO), cisplatin (twice week, 1 mg/kg), GSK-126 (3-times a week, 50 mg/kg), atorvastatin (ATR, 3-times a week, 20 mg/kg) or GSK-126 plus atorvastatin. FIG. 31b: Graph recapitulates the correlations between the time in days and the tumor volume in mm3. Two-way ANOVA (vehicle: n=8; cisplatin: n=7 ATR: n=7; GSK-126: n=7; Combo: n=8; p<0.0001), Tukey multiple comparisons post-test. FIG. 31c: Images of tumors extracted from euthanatized mice at Day 28. FIG. 31d: Blood circulating levels of ASAT, ALAT, creatinine and urea in the different groups of mice. ns, not significant; *, p<0.05; ** p<0.01; *** p<0.001.

### DETAILED DESCRIPTION

The present invention thus relates to methods of treating and/or preventing cancers as well as synergistic compositions and combinations for use in a method of treating and/or preventing cancers and tumors associated with histone methyltransferase EZH2 comprising administering to a subject in need a therapeutically effective amount of the composition, wherein said composition comprises the combination of EZH2 inhibitors and one or more statins. Said synergistic compositions or combinations are referred herein after as bi-therapies or combinatory therapies.

EZH2 inhibitors are typically S-Adenosyl-L-methionine (SAM) competitive inhibitors of EZH2 and carry 2-pyridone moiety or tetramethylpiperidinobenzamide moiety as described inter alia in Fioravanti R. et al. (Chem. Rec. 2018, 18, 1818-1832).

By way of example and without any limitations, EZH2 inhibitors may include catalytic 2-pyridone EZH2 inhibitors with a bicyclic heteroaromatic ring as the central scaffold, such as GSK-126 or GSK2816126 (1-[(2S)-butan-2-yl]-N-[(4,6-dimethyl-2-oxo-1H-pyridin-3-yl)methyl]-3-methyl-6-[6-(1-piperazinyl)-3-pyridinyl]-4-indolecarboxamide), UNC1999 (N-[(6-methyl-2-oxo-4-propyl-1H-pyri din-3-yl)methyl]-1-propan-2-yl-6-[6-(4-propan-2-ylpiperazin-1-yl) pyridin-3-yl]indazole-4-carboxamide), EPZ005687 (1-cyclopentyl-N-[(4,6-dimethyl-2-oxo-1H-pyridin-3-yl)methyl]-6-[4-(morpholin-4-ylmethyl)phenyl]indazole-4-carboxamide), and EI1 (6-cyano-N-[(4,6-dimethyl-2-oxo-1H-pyridin-3-yl)methyl]-1-pentan-3-ylindole-4-carboxamide). We can also cite MC3629, a simplified analogue of EPZ005687 and GSK-126, comprising pyrazole- and pyrrole-based compounds linked through an amide bond to the 2-pyridone moiety, GSK926 (N-[(4,6-dimethyl-2-oxo-1H-pyridin-3-yl)methyl]-6-[6-(4-methylpiperazin-1-yl) pyridin-3-yl]-1-propan-2-ylindazole-4-carboxamide), GSK343 (N-[(6-methyl-2-oxo-4-propyl-1H-pyridin-3-yl)methyl]-6-[2-(4-methylpiperazin-1-yl) pyridin-4-yl]-1-propan-2-ylindazole-4-carboxamide), and GSK503 (N-[(4,6-dimethyl-2-oxo-1H-pyridin-3-yl)methyl]-3-methyl-6-[6-(4-methylpiperazin-1-yl) pyridin-3-yl]-1-propan-2-yl indole-4-carboxamide). We can further cite CPI-360 carrying a 2-pyridone moiety linked to the indole nucleus through an amide function, its analog CPI-169 and CPI-1205 or Lirametostat (N-[(4-methoxy-6-methyl-2-oxo-1H-pyridin-3-yl)methyl]-2-methyl-1-[(1R)-1-[1-(2,2,2-trifluoroethyl) piperidin-4-yl]ethyl]indole-3-carbo xamide).

We may also cite Tazemetostat also named EPZ-6438 or E7438 (N-[(4,6-dimethyl-2-oxo-1H-pyridin-3-yl)methyl]-3-[ethyl(oxan-4-yl)amino]-2-methyl-5-[4-(morpholin-4-ylmethyl)phenyl]benzamide, as well as two orally available benzamidomethyl-2-pyridone analogues of tazemetostat: EPZ011989 which is also designated 1598383-40-4 (N-[(4,6-dimethyl-2-oxo-1H-pyridin-3-yl)methyl]-3-[ethyl-[4-[2-methoxyethyl(methyl)amino]cyclohexyl]amino]-2-methyl-5-(3-morpholin-4-ylprop-1-ynyl)benzamide) and ZLD1039 also designated 1826865-46-6 (3-[ethyl(oxan-4-yl)amino]-2-methyl-N-[(1-methyl-3-oxo-5,6,7,8-tetrahydro-2H-isoquinolin-4-yl)methyl]-5-[6-(4-methyl piperazin-1-yl) pyridin-3-yl]benzamide). Other analogs of tazemetostat include EBI-2511 (N-[(4,6-dimethyl-2-oxo-1H-pyridin-3-yl)methyl]-5-ethyl-6-[ethyl(oxan-4-yl)amino]-2-(1-propan-2-ylpiperidin-4-yl)-1-benzofuran-4-carboxamide), pinometostat ((2R,3R,4S,5R)-2-(6-aminopurin-9-yl)-5-[[[3-[2-(6-tert-butyl-1H-benzimidazol-2-yl)ethyl]cyclobutyl]-propan-2-ylamino]methyl]oxolane-3,4-diol), lirametostat (N-[(4-methoxy-6-methyl-2-oxo-1H-pyridin-3-yl)methyl]-2-methyl-1-[(1R)-1-[1-(2,2,2-trifluoroethyl) piperidin-4-yl]ethyl]indole-3-carboxamide).

We may further cite JQEZ5 also named JQE5 (1-isopropyl-N-((6-methyl-2-oxo-4-propyl-1,2-dihydropyridin-3-yl)methyl)-6-(6-(4-methylpiperazin-1-yl) pyridin-3-yl)-1H-pyrazolo[3,4-b]pyridine-4-carboxamide), PROTAC (PROteolysis TArgeting Chimeric) MS1943 (See, Feral et al., Adv. Therap. 2020, 3, 2000148), DZNep ((1S,2R,5R)-5-(4-aminoimidazo[4,5-c]pyridin-1-yl)-3-(hydroxymethyl)cyclopent-3-ene-1,2-diol)), MC1947, and MC1948.

Furthermore, we may cite PF-06821497 compound which is a catalytic EZH2 inhibitor comprising a 3,4-dihydroisoquinoline moiety in addition to the 2-pyridone moiety. The full chemical name is 5,8-dichloro-2-[(4-methoxy-6-methyl-2-oxo-1H-pyridin-3-yl)methyl]-7-[(R)-methoxy (oxetan-3-yl)methyl]-3,4-dihydroisoquinolin-1-one.

Other selective EZH2 inhibitors which may be used in synergistic bi-therapy and tri-therapy compositions according to the present invention include:
5.8-dichloro-2-[(4-methoxy-6-methyl-2-oxo-1,2-dihydro-pyridin-3-yl)methyl]-7-[methoxy (oxetan-3-yl)methyl]-3,4-dihydroisoquinolin-1 (2/-/)-one;
5.8-dichloro-2-[(4-methoxy-6-methyl-2-oxo-1,2-dihydro-pyridin-3-yl)methyl]-7-[(R)-methoxy (oxetan-3-yl)methyl]-3,4-dihydroisoquinolin-1 (2H)-one;
5.8-dichloro-2-[(4-methoxy-6-methyl-2-oxo-1,2-dihydro-pyridin-3-yl)methyl]-7-[(S)-methoxy (oxetan-3-yl)methyl]-3,4-dihydroisoquinolin-1 (2/-/)-one;
5-bromo-8-chloro-2-[(4,6-dimethyl-2-oxo-1,2-dihydropyridin-3-yl)methyl]-7-(1,4-dimethyl-f/-/-1,2,3-triazol-5-yl)-3,4-dihydroisoquinolin-1 (2/-/)-one; 5, 8-dichloro-7-(3,5-dimethyl-1,2-oxazol-4-yl)-2-[(4,6-dimethyl-2-oxo-1,2-dihydropyridin-3-yl)methyl]-3,4-dihydroisoquinolin-1 {2H)-one;
N-[(4,6-dimethyl-2-oxo-1,2-dihydropyridin-3-yl)methyl]-5-[ethyl(tetrahydro-2H-pyran-4-yl)amino]-4-methyl-4′-(morpholin-4-ylmethyl) biphenyl-3-carboxamide;
N-[(4,6-dimethyl-2-oxo-1,2-dihydropyridin-3-yl)methyl]-5-[ethyl(tetrahydro-2H-pyran-4-yl)amino]-4-methyl-4′-(morpholin-4-ylmethyl) biphenyl-3-carboxamide;
N-[(4-methoxy-6-methyl-2-oxo-1, 2-dihydropyridin-3-yl)methyl]-2-methyl-1-[(1R)-1-[1-(2,2,2-trifluoroethyl) piperidin-4-yl]ethyl]-1H-indole-3-carboxamide;
N-(4,6-dimethyl-2-oxo-1, 2-dihydropyridin-3-ylmethyl)-1-isopropyl-3-methyl-6-[6-(4-methylpiperazin-1-yl) pyridin-3-yl]-1H-indole-4-carboxamide;
N-[(6-methyl-2-oxo-4-propyl-1,2-dihydropyridin-3-yl)methyl]-6-[2-(4-methy lpiperazin-1-yl) pyridin-4-yl]-1-(propan-2-yl)-1H-indazole-4-carboxamide.

Said one statin may be elected among the group consisting of atorvastatin, fluvastatin, pitavastatin, pravastatin, mevastatin, rosuvastatin, simvastatin, cerivastatin, and/or analogues thereof. Statins are available upon prescription. There are several more under clinical investigation. Said one statin used in bi-therapies according to the present invention however preferably does not comprise lovastatin and thus is devoid of any lovastatin.

Statins are a group of compounds that are 3-hydroxy-3-methyl-glutaryl-coenzyme A reductase inhibitors (also known as HMG-COA inhibitors or HMGCR inhibitors) that function as lipid-lowering compounds. HMG-COA is an enzyme {i.e., NADH-dependent (EC 1.1.1.88), NADPH-dependent (EC 1.1.1.34)) that is the rate-limiting enzyme of the mevalonate pathway, i.e., the metabolic pathway that produces cholesterol and other isoprenoids. HMG-COA is normally suppressed by cholesterol that is derived from the internalization and degradation of low-density lipoprotein (LDL) via the LDL receptor, as well as oxidized species of cholesterol. Competitive inhibition of HMG-COA by statins initially reduces cholesterol production, and, as an adaptive response, the reduced cellular cholesterol level triggers the sterol regulatory element-binding protein (SREBP)-mediated activation of gene expression, including that of LDL receptors (LDLRs) in the liver and other tissues, which leads to an increased uptake of LDL from the circulation, hence lowering blood cholesterol levels.

Synergistic compositions or bi-therapies according to the present invention are effective for treating and/or preventing cancers and tumors associated with histone methyltransferase EZH2. They have been showed to produce beneficial changes in a patient's cancer status. The changes can be either subjective or objective and can relate to features such as symptoms or signs of the cancer being treated, such as reducing the number of cancer cells, the growth of the cancer cells, the size of cancer tumors, the resistance of the cancer cells to another cancer drug, and/or preventing the deterioration of the patient's status. In addition, these bi-therapies enabled administering reduced dose regimen of EZH2 inhibitors compared to previous clinical trials, thus allowing patients to suffer from less toxic side effects of the EZH2 inhibitors, but still retaining therapeutic efficacy against their cancers. The dosage of EZH2 inhibitor, such as GSK-126, may be reduced by at least half of the dose as compared to the dose currently used in clinical trials. Therefore, dose regimen of GSK-126 in the bi- or tri-therapies according to the present invention may be between 20 and 1500 mg, preferably between 50 and 1200 mg, and more preferably between 100 and 1000 mg twice weekly via intravenous administration.

In effect, Applicants showed herein below that administering the combinations of one EZH2 inhibitor such as GSK-126 with at least one statin and/or one or more anticancer drugs or chemotherapeutic agents sensitize the cancers or tumors to GSK-126, thereby reversing or reducing the resistance of patients to GSK-126.

As used herein, patient or subject may be used interchangeably and refers to a human in need of cancer treatment or at risk of developing a cancer/tumor.

Cancers and tumors associated with histone methyltransferase EZH2 may be selected in particular among hepatoblastoma (HB), Diffuse Intrinsic Pontine Glioma (DIPG, also known as “diffuse midline glioma H3K27M-mutant” because of the frequency of mutations in the histone H3 gene which are involved in the emergence of the disease; Louis, D. N., et al., The 2016 World Health Organization Classification of Tumors of the Central Nervous System: a summary. Acta Neuropathol, 2016. 131 (6): p. 803-20), Diffuse Midline Glioma (DMG), bladder cancer, bone cancer, brain cancers, breast cancer, malignant lymphoma, rhabdoid tumor, leukemia, lung cancer, stomach cancer, prostate cancer, colorectal cancer, oesophageal cancer, ovarian cancer, uterine cancer, liver cancer, testicular cancer, pancreatic cancer, renal cancer, rectal cancer, thyroid cancer, skin cancer, head & neck cancer. Said tumors and cancers are preferably not associated with endothelial to mesenchymal transition associated pathology.

Synergistic bi-therapy compositions or combinatory therapies according to the present invention are preferably administered to relapsed or refractory patients after anticancer standard treatment, radiotherapy or other first or second line of cancer therapies, as some of these cancers or tumors may be resistant to certain anti-cancer drug.

Indeed, as demonstrated by the Applicants in the Examples below, said bi-therapies have a significant synergistic therapeutic effect: the therapeutic and/or the beneficial effects that are produced when GSK-126 and one statin were administered in combination or co-administered were greater compared to when they were administered alone and greater to the effects produced by a sum of the effects of the individual compounds (i.e., an effect that is greater than an additive effect). In particular, an unexpected efficacy of the combinatory therapy or bi-therapy has been evidenced on cell migration and adhesion. Methods of identifying synergistic effects are discussed in various publications such as Foucquier J. et al. (Pharmacology Research & Perspectives (2015) (3) 3: e00149).

Because of the synergistic effects of the bi-therapy compositions, therapeutically or prophylactically effective amounts of EZH2 inhibitor which have been administered or co-administered in combination with one statin to a patient may be much lower than the doses administered so far in current clinical phases. Such reduced effective dosages of GSK-126 may be comprised between 1 and 20 mg/kg or between 5-10 mg/kg of the patient. Such reduction of the dosage regimen of EZH2 inhibitor within the bi-therapy of the present invention has a significant impact in greatly reducing the general toxicity of the drug to patient, while maintaining efficient anti-cancerous effects including the delay or inhibition of the cancer cell, the inhibition of the tumor growth, of the tumor vascularization, progression and/or metastasis, the reduction of tumor size, the induction of cell death, the increase of the mean time of survival, and the sensitization of cancer cell to an anticancer drug to which it has become or is resistant.

Synergistic compositions and methods of treating comprising the combination of GSK-126 and a statin according to the present invention were showed to eradicate hepatoblastoma cells in vitro, and to block hepatoblastoma growth and vascularization in vivo. These compositions are also effective in treating cisplatin-resistant hepatoblastoma.

In particular, Applicants clearly demonstrated in the Examples 2 and 4 that the methyl-transferase EZH2 is a key oncogene in hepatoblastoma and a relevant therapeutic target for the treatment of this pediatric liver tumor. Applicants demonstrated that EZH2 acted on one hand as a key oncogene in proliferative hepatoblastoma through its methyl transferase activity on histone H3 and non-histone targets, thereby resulting in chromatin condensation and the inhibition of tumor suppressor genes. Indeed, Applicants clearly showed that EZH2 methyltransferase activity was required for cell migration, survival, cisplatin resistance and tumor development and vascularization, and thus that EZH2 methyltransferase activity was central in the cancerous processes and the drug resistance. On the other hand, EZH2 was showed to act as a transcriptional cofactor inducing the overexpression of the HMG-COA reductase, which is responsible for cholesterol and lipids synthesis, and of DUSP9, a dual-specificity phosphatase involved in aggressive hepatoblastoma (See Khoubai and Grosset, Int J Mol Sci 2021, https://doi.org/10.3390/ijms222111538).

Regarding the synergistic effects of the bi- and tri-therapies, Applicants showed that HGMCR protein was induced in hepatoblastoma cells treated with GSK-126 suggesting a functional connection between EZH2 and Hydroxy-3-Methylglutaryl-CoA Reductase (HMGCR) genes. While for example, two statins Atorvastatin and Simvastatin have been widely used for the treatment of heart diseases and dyslipidemia, at the same doses, none of these statins had an effect on hepatoblastoma. However, it was surprisingly found that these statins actually increased the sensitivity of hepatoblastoma to inhibitors of EZH2 such as GSK-126, or EPZ6438. In addition, the sensitivity of hepatoblastoma to Atorvastatin and Simvastatin was increased by the addition of GSK-126, used at 50% of IC₅₀ dose, and hepatoblastoma died even more rapidly when adding cisplatin, used at 25% of IC₅₀ dose to the bi-therapy.

In the same manner, Applicants showed that there is an increased expression of the genes involved in lipid and cholesterol synthesis in DIPG treated with GSK126. In particular, an induction of the expression of the hydroxymethylglutary-CoA synthase (HMGCS), 3-Hydroxy-3-Methylglutaryl-CoA Reductase (HMGCR), Low Density Lipoprotein Receptor (LDLR), Niemann-Pick C1 protein (NP (1) and Squalene Epoxidase (SQLE) genes, and HMGCR and SQLE proteins, thereby demonstrating that GSK-126 activates the lipid and cholesterol synthesis pathway in DIPG.

This is the mechanistic explanation and confirmation to the synergistic effect of the bi-therapies compositions comprising EZH2 inhibitor with at least a statin which has been evidenced in the below Examples. As a result, hepatoblastoma and DIPG were less prone to escape drug effect. Furthermore, EZH2 inhibitors may be used alone or in combination with cisplatin to improve the management of hepatoblastoma and that the addition of a statin boosted the effect of GSK-126 by blocking the mevalonate pathway.

Using genetic or drug-based pharmacological approaches to inactivate EZH2 functions, Applicants showed that EZH2 protein is required for the 2D and 3D growth and survival of hepatoblastoma cells in vitro and the development of aggressive and angiogenic tumor in the chick embryo model.

Applicants further found that the depletion of EZH2 increased the sensitivity of hepatoblastoma cells to cisplatin and that the combination of cisplatin with GSK-126 had an additive effect on the elimination of tumoral cells in vitro. Used alone, GSK-126 was also very efficient to kill hepatoblastoma cells in vitro at concentrations ranging from 5 to 10 μM. These data thus clearly showed that such bi-therapies comprising for example cisplatin and GSK-126 was efficient in first line of treatment could improve the chemotherapy efficiency, and useful as a second line of treatment for patients in relapse and presenting an acquired resistance to cisplatin.

In addition, Applicants implemented one tri-therapy according to the present invention comprising an EZH2 inhibitor, a statin (such as for example Simvastatin or Atorvastatin) and cisplatin and showed that the antitumor potential of GSK-126 alone was also increased by the addition of Simvastatin or Atorvastatin, thereby demonstrating the therapeutic benefit of the tri-therapies.

Tri-therapies compositions according to the present invention comprise the bi-therapies composition as described herein above and further comprise one or more anticancer drugs may be chosen among platinum compounds or platinum-based agents, tyrosine kinase inhibitors, taxane derivatives, topoisomerase inhibitors, hormone therapeutic agents, anti-androgen drugs, androgen receptor agents, anti-angiogenesis agents, immunotherapeutic preparations, anti-inflammatory drugs, radiotherapeutic agents, biological preparations having anticancer effects, anticancer preparations made from natural substances.

Preferred anticancer drug used in the tri-therapies of the present invention are platinum compounds or platinum-based agents, such as for example cisplatin, carboplatin, and oxaliplatin, satraplatin, picoplatin, nedaplatin, triplatin, and/or lipoplatin. Applicants demonstrated in the Examples below that administering tri-therapy combinations comprising an effective amount of GSK-126, statins with a further anticancer drug, such as said platinum compounds, provide superior therapeutic effects, thereby resulting in a substantial inhibition cancer cell growth, inhibition cancer cell metastasis, decrease tumor size, increased survival time of the subject, and more generally in a substantial improvement of one or more signs and/or symptoms of cancer. It is possible that such tri-therapy combinations reverse or reduces cancer cell resistance to the anticancer drug, and/or sensitize cancer cells to the anticancer drug, but the mechanism of action is unknown.

Other chemotherapeutic agents which may be used in such tri-therapies may include but are not limited to anthracenediones (anthraquinones) such as anthracyclines (e.g, daunorubicin (daunomycin; rubidomycin, doxorubicin, epirubicin, idarubicin, and valrubicin); tamoxifen and metabolites thereof such as 4-hydroxytamoxifen (afimoxifene) and N-desmethyl-4-hydroxytamoxifen (endoxifen); taxanes such as paclitaxel (taxol), docetaxel, cabazitaxel, hongdoushan A, hongdoushan B, hongdoushan C, baccatin I and baccatin II; alkylating agents (e.g., nitrogen mustards such as mechlorethamine (HN2), cyclophosphamide, ifosfamide, melphalan (L-sarcolysin), and chlorambucil); ethylenimines and methylmelamines (e.g, hexamethylmelamine, thiotepa, alkyl sulphonates such as busulfan, nitrosoureas such as carmustine (BCNU), lomustine (CCNLJ), semustine (methyl-CCN-U), and streptozoein (streptozotocin), and triazenes such as decarbazine (DTIC; dimethyltriazeno-imidazolecarboxamide); antimetabolites (e.g, folic acid analogs such as methotrexate (amethopterin), pyrimidine analogs such as fluorouracil (5-fluorouracil; 5-FU), floxuridine (fluorodeoxyuridine; FUdR), and cytarabine (cytosine arabinoside), and purine analogs and related inhibitors such as mercaptopurine (6-mercaptopurine; 6-MP), thioguanine (6-thioguanine; 6-TG), and pentostatin or 2′-deoxycofonnycin); natural products, e.g, vinca alkaloids, such as vinblastine (VLB) and vincristine, epipodophyllotoxins such as etoposide and teniposide; antibiotics such as dactinomycin (actinomycin D), bleomycin, plicamycin (mithramycin), and mitomycin (mitomycin Q); enzymes such as L-asparaginase; substituted ureas such as hydroxyurea; methyl hydrazine derivatives such as procarbazine (N-methylhydrazine; MIH); and/or adrenocortical suppressants such as mitotane and aminoglutethimide.

Anti-androgen drugs are drugs which alter the androgen pathway by blocking the androgen receptors, competing for binding sites on the cell's surface, or affecting or mediating androgen production. Anti-androgen drugs include, but are not limited to, enzalutamide, abiraterone, bicalutamide, flutamide, nilutamide, apalutamide, finasteride, dutasteride, alfatradiol, and combinations thereof.

Radiotherapeutic agents are well known in the art and comprise external-beam radiation therapy and/or internal radiation therapy. External beam radiation therapy delivers radioactive beams of high energy X-rays and/or gamma rays to a patient's tumor, whereas internal radiation therapy delivers radioactive atoms to a patient's tumor. Both external beam radiation therapy and internal radiation therapy are used to suppress tumor growth or kill cancer cells by delivering a sufficient quantity of radioactivity to the target site. In some embodiments, the radiotherapeutic agent comprises a radioactive atom and is complexed with a biologic or synthetic agent to increase delivery to the target site. Radiotherapeutic agents may thus be coupled to targeting moieties, such as antibodies, to improve the localization of radiotherapeutic agents to cancerous cells.

Said tri-therapy compositions preferably do not comprise any inhibitors of RORy (Retinoic acid receptor-related orphan receptor y), do not comprise any anti-VEGF agent such as sunitinib, or agents such as gefitinib, erlotinib, sorafenib, sunitinib, dasatinib, lapatinib, nilotinib, bortezomib, or salinomycin, do not comprise VEFG/VEGFR inhibitors, do not comprise BCL2 inhibitors or LSD1 inhibitors.

The present invention thus also provides a composition for use in a method of treating and/or preventing tumors associated with methyltransferase EZH2, comprising administering to a subject in need a therapeutically effective the bi-therapy or tri-therapy as described above, wherein said bi-therapy or tri-therapy is administered to relapsed or refractory patients after anticancer standard treatment, radiotherapy or other first or second line of cancer therapies.

According to another aspect, the present invention provides a composition for use in a method of treating and/or preventing C2A subgroup hepatoblastoma characterized inter alia by EZH2 and/or topoisomerase 2-alpha (TOP2A) upregulation, comprising administering a therapeutically effective amount of an EZH2 inhibitor to a subject in need thereof.

In addition, the present invention provides a composition for use in a method of treating and/or preventing DIPG characterized inter alia by EZH2 and/or TOP2A upregulation, comprising administering a therapeutically effective amount of an EZH2 inhibitor to a subject in need thereof.

Treated subjects have all ages, but the majority of them have between 1 and 5 years old.

According to this aspect of the invention, GSK-126 is preferred as EZH2 inhibitor, since it is 20 times less toxic than cisplatin which has been used until now as the gold standard drug in the first-line treatment of hepatoblastoma but was known as highly toxic and as causing serious secondary pathologies. Therefore, according to this aspect, GSK-126 was used as a new therapeutic option in proliferative and aggressive hepatoblastoma or in the second-line therapy for patients in relapse or presenting chemoresistance to cisplatin.

Indeed, Applicants demonstrated in Example 3 that EZH2 upregulation was associated with the presence of particularly aggressive hepatoblastoma (HB) and constituted a novel unfavorable prognostic factor, allowing to predict the outcome of the patients such as higher risks of relapse or death. Such upregulation has been evidenced in one subgroup of HB tumor designated as C2A subgroup which is one of the three subgroups of HB tumors, named C2A and C2B, having the poorest prognosis, more advanced tumor stage and worst overall survival rate. The transcript profiling separated HB into three distinct subgroups named C1, C2A, and C2B, identifiable by a concise four-gene signature: hydroxysteroid 17-beta dehydrogenase 6, integrin alpha 6, topoisomerase 2-alpha, and vimentin, with topoisomerase 2-alpha (TOP2A) being characteristic for the proliferative C2A tumors (See Hooks K B et al., Hepatology, 2018 July; 68 (1): 89-102. doi: 10.1002/hep.29672). Applicants further demonstrated that the highly proliferative tumor C2A subtype was not only characterized by topoisomerase 2-alpha gene up-regulation, but also by EZH2 upregulation which was showed to be positively and strongly correlated with the upregulation of TOP2A. Upregulation of EZH2 and/or TOP2A is an indicator of the particularly aggressive nature of the tumor and of high proliferation rate of the cancerous cells, with a poor outcome for the patient of relapse or death.

It was showed that EZH2 is upregulated in HB and its expression is significantly increased in tumors with unfavourable molecular and clinical biomarkers including TOP2A-expressing cells and patients in relapse or dead. EZH2 may thus be used as independent prognosis factor in HB or DIPG for relapse or death of the patients. Alternatively, the combination of EZH2 and TOP2A, or EZH2 and DUSP9 (Dual Specificity Phosphatase 9), or EZH2, TOP2A, and DUPS9 may thus be used as prognosis factors in HB or DIPG for relapse or death of the patients.

According to this aspect, the present invention thus provides use of EZH2 as a biomarker of poor prognosis with a higher likelihood of relapse and even death of patients suffering from HB or DIPG, and to a method of assessing and/or predicting the prognosis of a patient suffering from cancers chosen among HB or DIPG comprising determining in vitro the level of EZH2 in an isolated sample of said subjects. When the level of EZH2 is equal or higher than a reference control value, a poor prognosis of HB or DIPG is determined. Typically, the reference control value is the serum level of EZH2 resulting from the median of levels of EZH2 of a cohort of subjects suffering from HB or DIPG. For example, the level of EZH2 is equal or higher.

According to this aspect, the method of predicting the prognosis of a subject suffering from HB or DIPG comprises (i) detecting the level and concentration of EZH2 at a protein level or RNA level in an isolated sample from said subject, and (ii) comparing the detection result to that of a corresponding biomarker from a control sample or to that of a reference value, wherein an increased concentration, increased levels of at least the biomarker EZH2 in said subject compared to the control or reference value is indicative of a negative evolution of the subject. In addition to assessing the biomarker EZH2, the method may further comprises assessing additional biomarker of negative clinical evolution of said patients, including for example assessing the TOP2A and/or DUSP9 biomarkers.

Within the same aspect, the present invention provides in vitro kits comprising an agent suitable for detecting the level, concentration and/or presence of at least EZH2 at the protein level or RNA level. Such kits may further comprise agents allowing to assess the level, concentration and/or presence of TOP2A and/or DUSP9 at the protein level or RNA level. These agents are well known in the art. Some are used to run assays for protein detection and quantification, such as for example western blot, ELISA, radioimmunoassay, immunodiffusion assay, immunoelectrophoresis, immunostaining, immunoprecipitation, mass spectrometry, and protein microarray. Other agents are suitable for detection of the overexpression of the biomarker EZH2 alone or in combination with TOP2A and/or DUSP9, at the RNA level.

The present invention still also provides cancer therapies which comprise a pre-screening step of the patients in order to detect overexpression of at one biomarker EZH2 alone or in combination with TOP2A and/or DUSP9, thereby allowing to plan for specific protocols of treatment and specific follow-ups of these patients.

The present invention further provides pharmaceutical compositions comprising the bi-therapy or tri-therapy combinations as described above and a pharmaceutically acceptable excipient and/or carrier and/or diluent. The pharmaceutical compositions may also optionally comprise one or more anticancer drugs. Any pharmaceutically acceptable carrier and/or excipient and/or diluent suitable for the formulation of the present composition and the desired administration is contemplated herein.

Typically, such pharmaceutically acceptable excipient may include a salt or a diluent, and may further comprise pharmaceutical cryoprotectant, such as glucose, sucrose, trehalose, lactose, sodium glutamate, PVP, HPpCD, CD, glycerol, maltose, mannitol, and saccharose.

The pharmaceutical compositions of the present invention include formulations suitable for topical, parenteral, pulmonary, nasal, rectal, or oral administration. The most suitable route of administration in any given case will depend in part on the nature and severity of the cancer condition and also optionally the stage of the cancer. Preferred pharmaceutical compositions are formulated for parenteral administration, and most preferably for intravenous administration. Both immediate release and sustained release dosage forms are within the scope of the present invention.

Pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent(s).

Pharmaceutical compositions are thus preferably formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions. Formulations may be sterilized and/or contain adjuvants, such as preserving, stabilizing, wetting or emulsifying agents, solution promoters, salts for regulating the osmotic pressure and/or buffers.

According to the present invention, the EZH2 inhibitor is advantageously co-administered with a statin and optionally anticancer agent to produce a synergistic effect on treatment and/or prevention of cancers and tumors. The EZH2 inhibitor, statins and as well as anticancer agent may be administered concomitantly (co-administered) or sequentially.

In case of tri-therapies, the EZH2 inhibitor and the statin may be administered to the patient in combination with an anticancer drug via the same or a different administration route, orally or parenterally (e.g, intravenously). For example, the EZH2 inhibitor and the satins are administered parenterally (e.g, intravenously), while the anticancer drug may be administered orally, or vice versa.

The patient may thus receive a therapeutically effective amount of GSK-126 compound and one statin. Such administration may include providing effective amounts for a specified period of time, e.g., for about 1 to 24 hours or 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days, or in a specified sequence, e.g., administration of GSK-126 and a statin followed by the administration of one or more anticancer drugs, or vice versa. The protocol of treatment may include sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g, sequentially or simultaneously, via intravenous or intraperitoneal injection.

Therefore, the pharmaceutical compositions may be prepared as a single medicament or separate medicaments comprising individual dosage units made by admixing the EZH2 inhibitor and one statin (e.g, atorvastatin, simvastatin, fluvastatin, rosuvastatin, mevastatin, cerivastatin, pravastatin, and/or pitavastatin), a pharmaceutically acceptable carrier and/or excipient or diluent, and optionally one or more anticancer drugs.

Pharmaceutical compositions or medicaments may be administered to a subject at a therapeutically effective dose to prevent, treat, sensitize, or control a cancer responsive to EZH2 inhibition. The pharmaceutical composition or medicament was administered to a subject in an amount sufficient to elicit an effective therapeutic response in the subject. An effective therapeutic response included a response that at least partially arrested or slowed the symptoms or complications of the cancer. An amount adequate to accomplish this was defined as a “therapeutically effective dose.”

The dosage of active agents administered is dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates. Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD50 (the dose lethal to 50% of the population) and the ED50 (the dose therapeutically effective in 50% of the population). The dose ratio between toxic and therapeutic effects is the therapeutic index and can be expressed as the ratio, LD50/ED50. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Generally, an efficacious or effective amount of a composition is determined by first administering a low dose or small amount of the composition, and then incrementally increasing the administered dose or dosages, adding a second or third medication as needed, until a desired effect of is observed in the treated subject with minimal or no toxic side effects.

Single or multiple administrations of the compositions are administered depending on the dosage and frequency as required and tolerated by the patient. In any event, the composition should provide an efficient amount of the compositions of this invention to effectively treat the patient. Generally, the dose is sufficient to treat or ameliorate symptoms or signs of disease without producing unacceptable toxicity to the patient.

Throughout this application, various references are referred to and disclosures of these publications in their entireties are hereby incorporated by reference into this application to more fully describe the state of the art to which this invention pertains.

### EXAMPLES

### Example 1: Bi-Therapies Efficiency for Treating and/or Preventing Diffuse Midline Glioma (DMG)

### Example 1.1: Methods

### Microarray Analysis

The GSE50021 expression profile (35 DMG samples, 10 normal brain samples), was extracted from the Gene Expression Omnibus database. Raw reads were quantile normalized and log 2 transformed. Expression values identified by ILMN_1652913 probe for EZH2 were extracted and analyzed using GraphPad Prism.

### DMG Cell Lines and Primary BXdmg1 Cells

DMG cell lines used in this study were NEM157i, NEM157i-VEGF, NEM163i, SU-DIPG-IVi, SU-DIPG-IVi-Luc and primary cells BXdmg1. Origin and lentiviral modification procedures (including immortalization) as well as culture conditions have been described herein below.

### Chemical Inhibitors

EZH2 inhibitor GSK-126, Atorvastatin, ACSS2 inhibitor and Terbinafine have been used (Selleckchemicals, Houston, USA).

### Western Blots

Blots were performed using standard techniques (see below).

### Histone Extraction

Histone extraction was performed according to manufacturer's instructions (Histone Extraction Kit-ab113476, Abcam, Paris, France).

### Proliferation Assays

Cell growth was measured with the In vitro Toxicology Assay kit (Sulforhodamine B, Sigma Aldrich, Saint Quentin Fallavier, France) according to manufacturer's instructions. Cells were plated at a density of 2000 cells per well in 96-well plates in triplicates. Absorbance was measured at 565 nm using the CLARIOstar multiplate reader (BMG Labtech, Champigny-sur-Marne, France) at indicated time points.

### Apoptosis Assays

After exposure to drugs at indicated concentrations and times, cells were labeled with Annexin V-PE (BD Biosciences, Le Pont de Claix, France) and 7-AminoActinomycin D (7-AAD) (BD Bioscience) and analyzed using Flow Cytometry as described [4].

### Migration Assays

2×104 cells/well were placed in a 96-well and incubated for 24h. An IncuCyte Wound Maker (96-pin wound making tool) was used to make scratches.

### Spheroid Cultures

Standard culture methods for spheroid cultures were used for both DMG cell lines and hepatoblastoma cell lines.

### DMG In Vivo Models

Animal procedures were carried out in agreement with the European (directive 2010/63/UE) and French (decree 2013-118) guidelines. Mouse experiments have been authorized by local ethic commission and validated by the French Minister of Higher Education, Research and Innovation (APAFIS #13466-2019032112211281, authorization number B33063916).

The DMG NEM157i/NEM157i-VEGF CAM model has been described before Capdevielle V et al. Neuro Oncol 2019; 10.1093/neuonc/noz215).

A murine orthotopic model (SU-DIPG-IVi-Luc in NOD/LtSz-scid IL2R gamma mice) was developed based on the work of Mohammed et al. (Nat Med 2017; 23 (4): 483-492).

### Chemical Inhibitors

GSK-126 is a highly selective EZH2 methyltransferase inhibitor with an IC50 of 9.9 nM, (>1000-fold selective for EZH2 over 20 other human methyltransferases). GSK-126 (SelleckChem) was dissolved with DMSO (Dimethyl sulfoxide) and stored at −20° C. Three inhibitors of the cholesterol synthesis pathway have been used: Atorvastatin, an inhibitor of HMG-COA reductase, ACSS2 inhibitor which targets acetate-dependent acetyl-CoA synthetase 2 (ACSS2) and Terbinafine (squalene epoxidase inhibitor). All inhibitors were purchased at Selleckchemicals (Houston, USA). All inhibitors were aliquoted in DMSO and stored at −20° C.

### Primary Cell Isolation, Culture and Characterization

All cell lines were tested regularly for the presence of mycoplasma and have been successfully STR profiled for authentication in April 2021 (LGC, Molsheim, France). Patients (or their guardians) enrolled for therapy at the University Hospital in Bordeaux gave written consent that biological probes can be used for research purposes in an anonymized manner, according to French national guidelines.

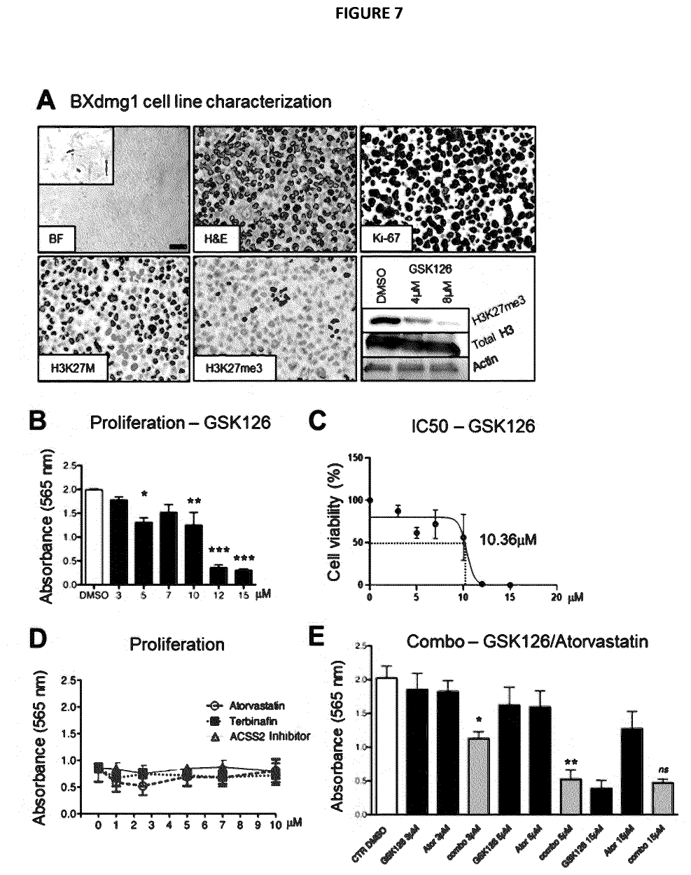

A DMG biopsy was received in the laboratory and dissociated using GentleMACs cell separator (Miltenyi Biotech, Paris, France) within 24h using MACSBrain Tumor Dissociation Kit (Miltenyi Biotech, Paris, France) and the standard GentleMACs brain tissue separation program. Cells yielded were cultivated in Amniomax C100SUP plus Amniomax C100 basal medium (Gibco, ThermoFisher, Illkirch Cedex, France). Molecular and cellular analysis of the biopsy were detailed in FIG. 7 (see Rahal, F., C. Capdevielle, B. Rousseau, J. Izotte, J. W. Dupuy, D. Cappellen, G. Chotard, M. Menard, J. Charpentier, V. Jecko, C. Caumont, E. Gimbert, C. F. Grosset and M. Hagedorn (2022). “An EZH2 blocker sensitizes histone mutated diffuse midline glioma to cholesterol metabolism inhibitors through an off-target effect.” Neurooncol Adv 4 (1): vdac018).

For characterization of biopsy-derived cells (BXdmg1) immunohistochemistry was used. Cells were harvested and included in CytoBlocks prior to sectioning and staining (FIG. 7A). Automated staining was performed on an Omnis Dako® stainer after heat antigen retrieval in citrate buffer, Flex amplification treatment and incubation with following antibodies: Ki-67 (Dako, clone MIB1, 1/100), H3-K27M (Diagomics, clone RM192, 1/5000) and H3-K27me3 (ABcam, clone 8290, 1/100). Revelation was done using diaminobenzidine (Dako). The cells were regularly passaged and displayed a homogeneous morphological phenotype, they have been termed BXdmg1.

### Western Blots

Cell lysates were prepared by scraping cells using RIPA buffer (Sigma Aldrich) plus proteinase inhibitors (Sigma Aldrich) and centrifuged at 13000 g for 15 min at 4° C. Protein concentrations were measured using Pierce™ BCATM Protein Assays (ThermoFisher) and equal amounts of cell extracts were loaded for western blot analysis in 4-15% precasted polyacrylamide gel (Bio-Rad). Proteins were blotted on a nitrocellulose membrane (Transblot® Turbo midi-size, Bio Rad), blocked with the Odyssey blocking buffer (LI-COR Biosciences, ScienceTec, Les Ulis, France) or with BSA diluted in Tris-buffered saline with 0.1% Tween 20 (TBST) and probed with primary antibodies overnight at 4° C. Primary antibodies used were: rabbit polyclonal anti-EZH2 (1:1000 dilution, #5246S, Ozyme/Cells signaling, Saint Cyr l'Ecole, France), rabbit anti-GAPDH (1:15000, BLE649203, Ozyme/Cells signaling), rabbit monoclonal H3K27me3 (1:1000, 9733S, Ozyme/Cells signaling), mouse monoclonal histone H3 (1:500, sc-517576, Santa-Cruz, Heidelberg, Germany), mouse monoclonal actin (C-2) (1:500, sc-8432, Santa-Cruz), mouse monoclonal anti-HMGCR (1:1000, CL0259, Atlas antibodies, Bromma, Sweden), rabbit polyclonal anti-SQLE (1:1000, 12544-1-AP, Proteintech, Manchester, UK), diluted in blocking buffer or 5% BSA. After washing with TBST, the membranes were incubated with corresponding goat anti mouse IgG (H+L)-HRP conjugate (1:3000, 170-6516, Biorad, Marnes-la-Coquette, France) or anti-rabbit IgG-HRP (1:3000, A0545, Sigma Aldrich, Saint Quentin Fallavier, France). After washing with TBST (twice for 10 minutes), membranes were revealed with Fusion FX (Vilber Lourmat). Quantification was performed using ImageJ (National Institutes of Health, Bethesda, Maryland, USA).

siRNA Transfections

siRNAs were diluted in the 1×siMAX dilution buffer (30 mM HEPES, 100 mM KCl, 1 mM MgCl2, pH=7.3 (Eurofins, Ebersberg, Germany). Cells were transfected independently with each EZH2 siRNA or control siRNA (AllStars Negative Control siRNA, Qiagen, Courtaboeuf, France). Briefly, 10 nM of siRNA was transfected with RNAiMAX transfection reagent (Invitrogen) according to the manufacturer's instructions for reverse and forward transfections. Prior to transfection, Lipofectamine RNAiMAX (ThermoFischer) was diluted 1/100th in transfection medium (OptiMEM, Gibco, ThermoFisher, Illkirch Cedex, France).

### Cell Migration Assays and Spheroid Cultures

Migration was monitored by using the IncuCyte S3 live-cell analysis system (Essen BioScience, Ltd, Royston Hertfordshire, United Kingdom). The original unmodified Incucyte images were used for statistical analysis but for demonstration of the effects images have been transformed using Adobe Photoshop CS4 (San Jose, USA). Following functions have been used to optimize visibility of the cells: greyscale mode, bichrome mode, negative, contrast/luminosity, exposition/gamma, negative.

For spheroid formation assays, 10⁴ cells were used per well in a 96-well plate. Volume of methylcellulose/medium/cells and inhibitors (10, 15 or 20 μM) mixture per well was 100 μL with a final concentration of methylcellulose at 0.5%. After rapid and gentle homogenization, the mixture was placed on a round-bottom cell culture microplate (U-shaped) treated to limit cell adhesion. After, spheroids were incubated in an incubator at 37° C. and 5% CO2 for 24 hours. Photos and films of spheroids were taken by an InCellis cell imager (Bertin instruments, Montigny-le-bretonneux, France). Since no dose-dependent effect was observed at the doses tested, all doses were pooled for statistical analysis.

### RNA Purification and Real-Time Quantitative PCR Analysis

Total RNA was isolated from cell lines with the TRI Reagent (Sigma) following manufacturer's instructions. For quantification of messenger RNA (mRNA) expression, total RNA was retrotranscribed using the Maxima Reverse Transcriptase (Thermo Scientific). RT-qPCR amplifications were performed in 12 μL multiplex PCR reactions containing 1×SYBR® Premix Ex Taq™ (Takara Bio Europe). Forward and reverse primers were as described in the Table 1. The GAPDH mRNA served as internal control for normalization.

TABLE 1
Primers used for real-time
quantitative PCR analysis

| | | |
|---|---|---|
| SEQ ID NO: 1 | SQLE- | 5′-GCCTGCCTTTCATTGGCTTC-3′ |
| | Forward | |
| SEQ ID NO: 2 | SQLE- | 5′-TTCCTTTTCTGCGCCTCCTG-3′ |
| | Reverse | |
| SEQ ID NO: 3 | NPC1- | 5′-ACTCAGTTACATAGGGCCATCA-3′ |
| | Forward | |
| SEQ ID NO: 4 | NPC-1- | 5′-CGACCGATCCTTAGACACAG-3′ |
| | Reverse | |
| SEQ ID NO: 5 | LDLR- | 5′-AGCTACCCCTCGAGACAGAT-3′ |
| | Forward | |
| SEQ ID NO: 6 | LDLR- | 5′-ACTCTCCGAAGCCTGTTCTG-3′ |
| | Reverse | |
| SEQ ID NO: 7 | HMGCR- | 5′-AGTGAGATCTGGAGGATCCAAG-3′ |
| | Forward | |
| SEQ ID NO: 8 | HMGCR- | 5′-ACAAAGACGCCATCCATTCG-3′ |
| | Reverse | |
| SEQ ID NO: 9 | HMGCS- | 5′-CTTGTGCCCGAAGCAGGAAA-3′ |
| | Forward | |
| SEQ ID NO: 10 | HMGCS- | 5′-GGCATGGTGAAACAGCTGTG-3′ |
| | Reverse | |

### Chicken and Mouse Models

For the chick CAM assay DMG model, 1×10⁶ NEM157i/NEM157i-VEGF (50:50) cells were mixed with Matrigel including inhibitors at indicated concentrations and 40 μl were put directly on the CAM. Tumor growth was monitored using a stereomicroscope (DS-Fi2, Nikon/SMZ745T) every two or three days. Tumors were fixed with PFA 4% and proceeded for photo documentation. A similar approach was used for hepatoblastoma Huh6 and HepG2 cells.

For the mouse model, the immortalized SU-DIPG-IV line was used. SU-DIPG-IVi was transduced by a luciferase vector (Luc) with a MOI of 10. Two days after birth, 10⁵ SU-DIPG-IVi-Luc cells in 2 μl were injected directly into the brainstem through the neck to a depth of 3 mm using a 2 μl NeuroSyringe (Hamilton Neuros, Dutscher, Bruxelles, Belgium) under anesthesia with 2% isoflurane and 50% oxygen enrichment. Treatment begun after 8 days, 3-times a week with solvent control (DMSO) or GSK-126 (6 or 10 mg/kg), Atorvastatin (10 mg/kg) or combo (statin and GSK-126 together). Tumor growth was evaluated non-invasively with anesthetized mice on a Biospace imager (Biospace Lab, Nesles la Vallée, France) 1-2 days after each treatment.

Prior to treatments, mice were micro-tattooed (Aramis kit, BiosebLab, France). Animals were weighed and then receive an i.p. injection in the lower right quadrant following IACUC recommendations. A 1 ml tuberculin syringe and a 26-gauge needle were used with an injection volume of 200 μl for a 30 g mouse (3 mg/ml), adjusted to actual weight each week. Treatment was done 3 times a week, starting at day 8. Tumor growth was evaluated non-invasively on a Biospace imager (Biospace Lab, Nesles la Vallée, France). The entire procedure was carried out on a heating mat at 37° C. The animals were shaved and anesthetized in sterile condition (under PSM-2) in boxes dedicated to imaging. Prior to imaging, animals received an i.p. injection of 150 mg/kg of D-Luc (Promega, E264X) diluted in PBS (50 to 100 μl), depending on the weight of the animal. The box was then placed on a heating mat at 37° C. in the photo imager in which 2% isoflurane with 50% oxygen is maintained. Imaging was done twice a week. Anesthesia time was around 15 minutes since only immobility was desired and awakening of the animals is almost instantaneous. At day 21, animals were killed by cervical dislocation.

### Statistical Methods

Statistical analyzes were performed with GraphPad Prism 5 software (GraphPad Software, Inc. San Diego, USA). For quantitative comparisons of more than two samples, One-way ANOVA test was used followed by Bonferroni post-test. If data distribution was not normal, Kruskal-Wallis test was used following Dunn's post-test of selected relevant conditions. For the comparisons of two small independent samples, unpaired t-test was used. For experiments analyzed by phenotypic evaluation using a semi-quantitative approach, Fisher's Exact test was used. All experiments (except in vivo experiments) were carried out, independently, at least 3 times, n=independent experiments. A p-value of <0.05 was considered to be statistically significant. For all data in figures, *: p<0.05, **: p<0.01, ***: p<0.001 or exact p-values where indicated.

### Example 1.2: Results

### EZH2 Gene and Protein Expression in DMG Samples and Cell Lines

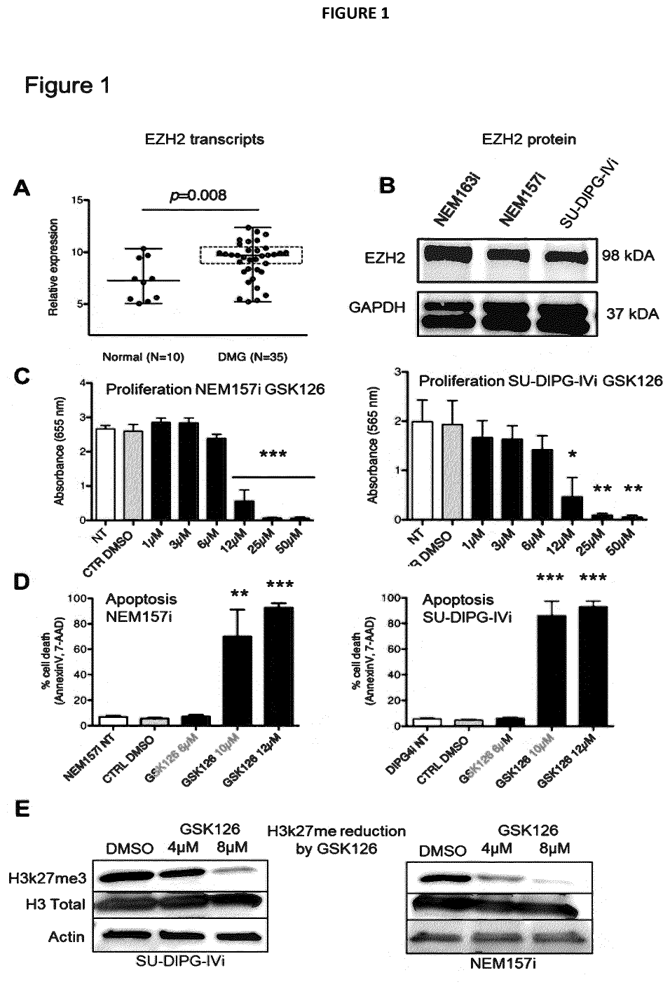

Transcripts of EZH2 were significantly over expressed in DMG samples compared to normal brain (FIG. 1A). Similar results have been found in other solid malignancies ranging from prostate, lung, hepatocellular, colorectal, breast to pancreatic cancer. Mean EZH2 over expression was not very elevated compared to controls due to high expression variability between the two groups, but a core set of samples regrouped around the median EZH2 expression and differences were still significant (FIG. 1A, left plot, boxed frame). Cell lines used in this study all showed expression of EZH2 protein as revealed by Western Blot (FIG. 1B).

### DMG Cells are Sensitive to GSK-126 Inhibitor

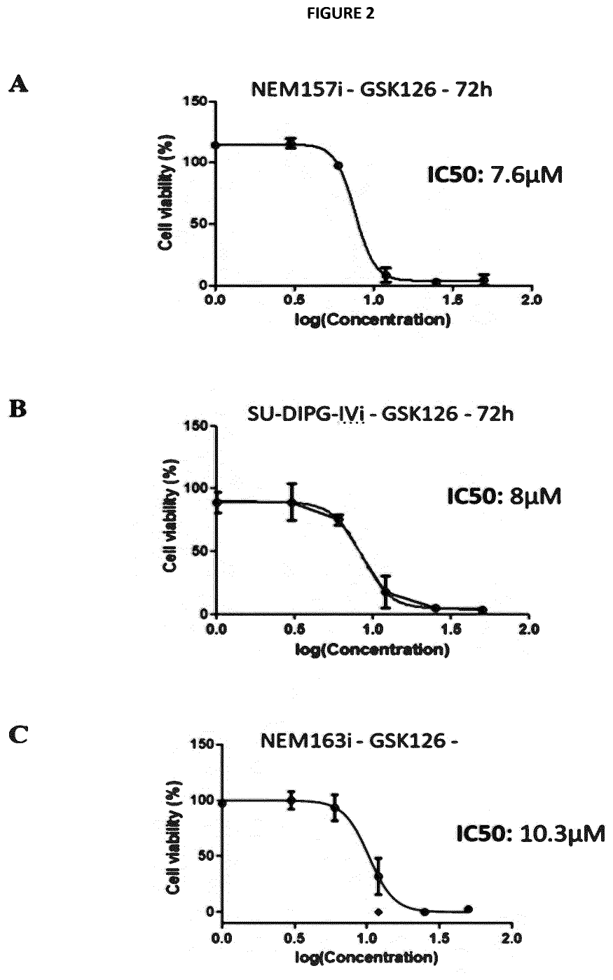

Significant growth inhibition of GSK-126 was observed at doses above 6 μM and was total at higher doses above 25 μM (FIG. 1C). Growth inhibition was accompanied by an increase in tumor cell apoptosis, at similar doses when cell proliferation was strongly reduced (FIG. 1D). Moreover, GSK-126 efficiently inhibited H3K27me3 trimethylation in DMG cells (FIG. 1E). Finally, the half maximal inhibitory concentration (IC50) of GSK-126 was measured in three DMG cell lines and ranged from 7.6 to 10.3 μM (FIG. 2).

### Increased Expression of Genes Involved in Lipid and Cholesterol Synthesis in GSK-126-Treated DIPG Cells

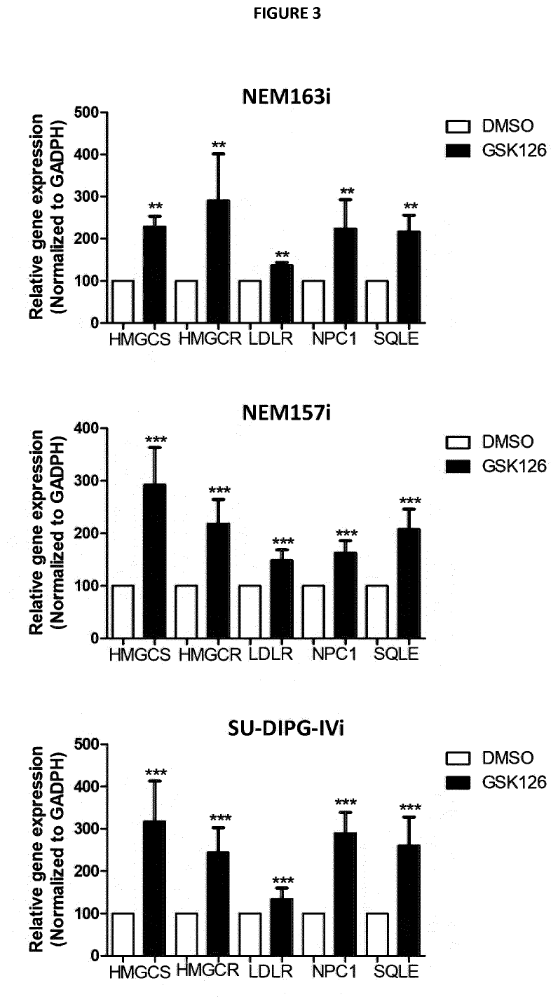

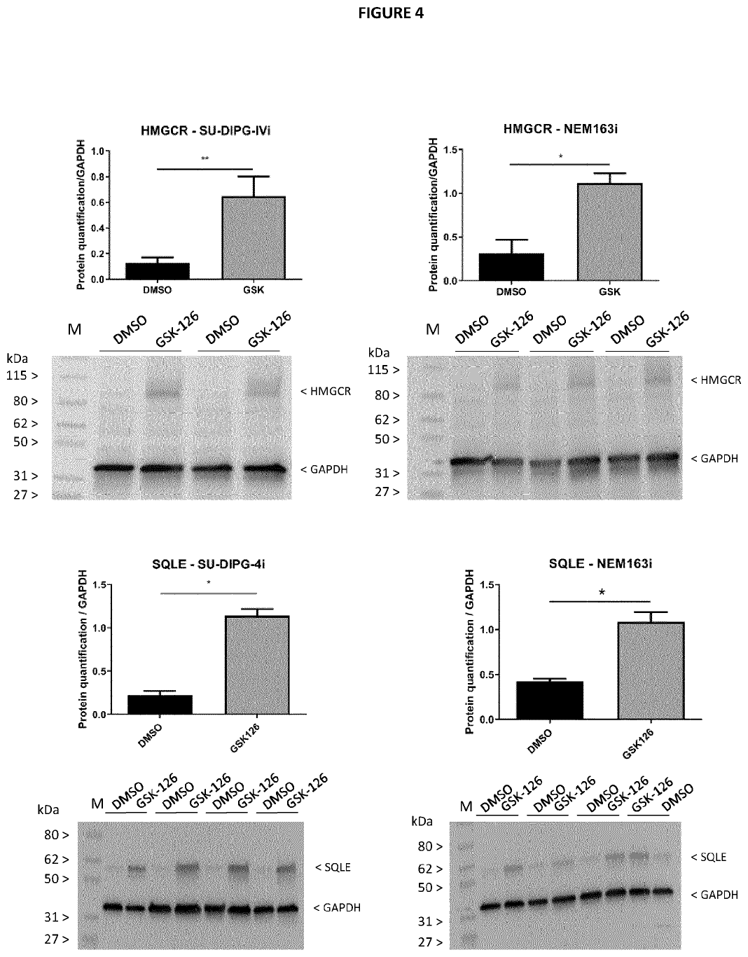

Treatment of DIPG cells by GSK-126 induced the expression of HMGCS, HMGCR, LDLR, NPC1 and SQLE genes, and HMGCR and SQLE proteins (FIGS. 3 and 4). These results confirm our proteomic data (see Rahal, F., C. Capdevielle, B. Rousseau, J. Izotte, J. W. Dupuy, D. Cappellen, G. Chotard, M. Menard, J. Charpentier, V. Jecko, C. Caumont, E. Gimbert, C. F. Grosset and M. Hagedorn (2022). “An EZH2 blocker sensitizes histone mutated diffuse midline glioma to cholesterol metabolism inhibitors through an off-target effect.” Neurooncol Adv 4 (1): vdac018), and further demonstrate that GSK-126 activates the lipid and cholesterol synthesis pathway in DIPG cells.

Effects of ACSS2 Inhibitor, Atorvastatin and Terbinafine on DMG Cells Alone or in Combination with GSK-126

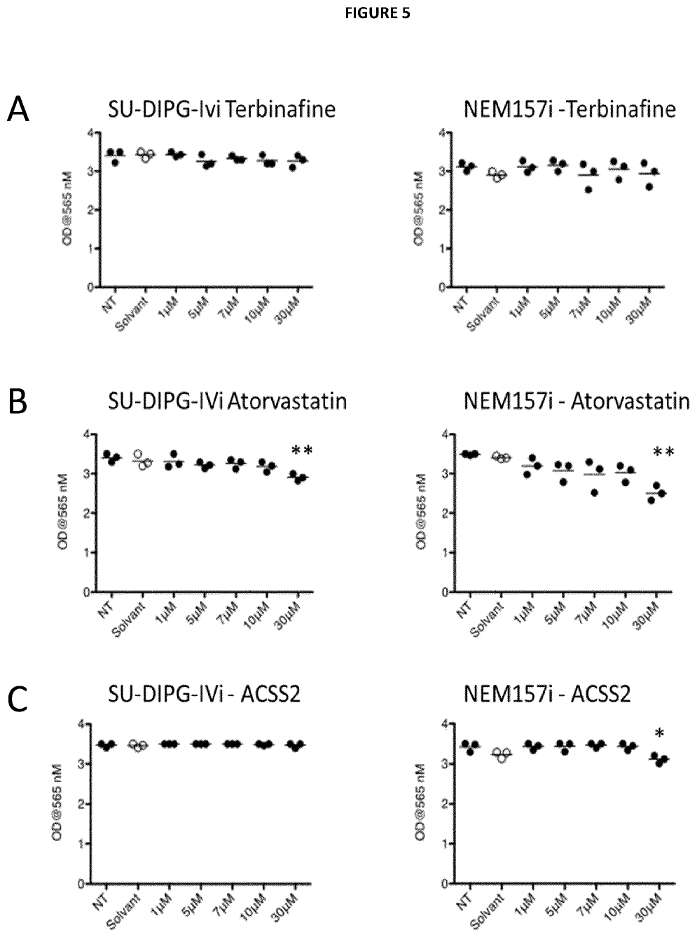

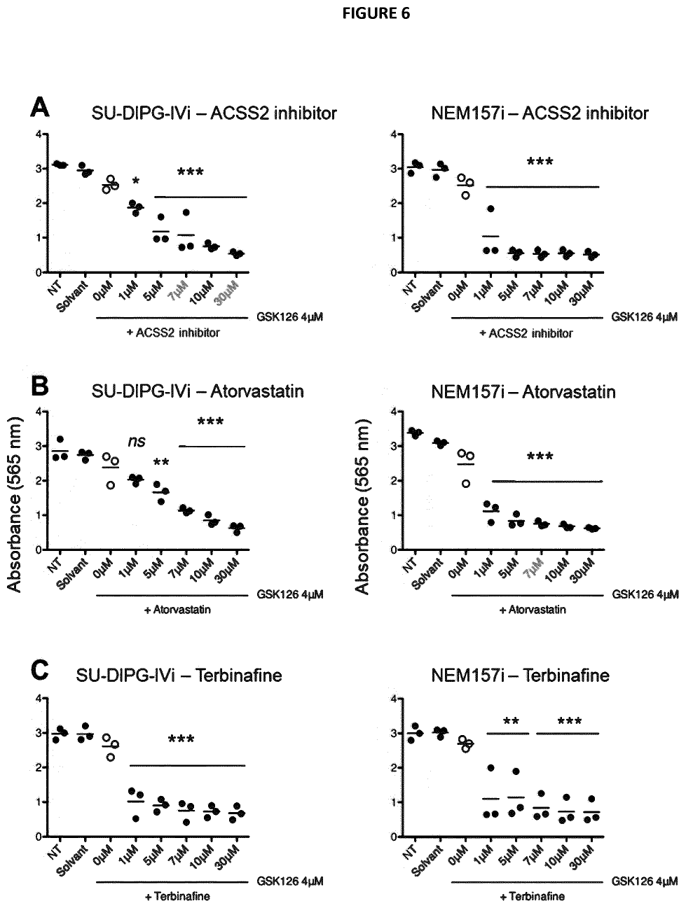

In order to interpret these changes, we hypothesized that several key enzymes involved in the biosynthesis of cholesterol [squalene epoxidase, hydroxymethylglutaryl-coenzyme A (HMG-CoA) reductase, AcetylCOA acetate-dependent acetyl-CoA synthetase 2] became important to GSK-126-treated DMG cells. These enzymes have been known for a long time to be implicated in diseases related to lipid metabolisms. For each cholesterol biosynthesis inhibitor tested, no effects have been observed on two different tumor cell lines alone (FIG. 5). However, when co-treated with a low dose of GSK-126 (4 μM), which does not affect DMG cell growth (FIG. 1C, FIG. 7D), significant growth inhibition occurred for all three inhibitors at doses ranging from 1 to 5 μM (FIG. 6, A-C).

### Validation of DMG Cell Line Data on Freshly Isolated DMG Cells

Cell lines which are immortalized or cultured for a certain time can behave differently as the original cells. We therefore isolated cells from a DMG biopsy for functional analysis. Bright field microscopy shows a homogenous, spindle-formed cell population (FIG. 7A, and insert). H&E staining of BXdmg1 cells revealed atypical eosinophilic cells with irregular anisocaryotic cell nuclei with very high mitotic activity, almost all tumor cells express the Ki-67 antigen (FIG. 7A). Tumor cells were also strongly positive for the H3K27M mutation and negative for H3K27me3 trimethylation (FIG. 7A). Cellular, molecular, and genetic characterization of the biopsy from which BXdmg1 cells originated, notably confirming the c83A>T; pK28M mutation in histone H3F3A, leading to the driver oncogenic event, which is conserved in BXdmg1 cells.

GSK-126 treatment reduced H3K27me3 trimethylation in a dose-dependent manner as revealed by Western blots of histone-purified protein extracts (FIG. 7A, lower right panel). BXdmg1 cells are sensible to growth inhibition by GSK-126 (FIG. 7B) with an IC50 around 10 μM (FIG. 7C), comparable to the other DMG lines. BXdmg1 cell growth is not affected by three cholesterol biosynthesis inhibitors, up to 10 μM (FIG. 7D). Combining GSK-126 and Atorvastatin showed significant growth inhibition at low doses of 3 and 5 μM for each inhibitor, this effect was masked, as expected, at higher doses because of already established cytotoxicity of GSK-126 alone (FIG. 7E).

### Effects of Atorvastatin, GSK-126 and Combination on Cell Migration

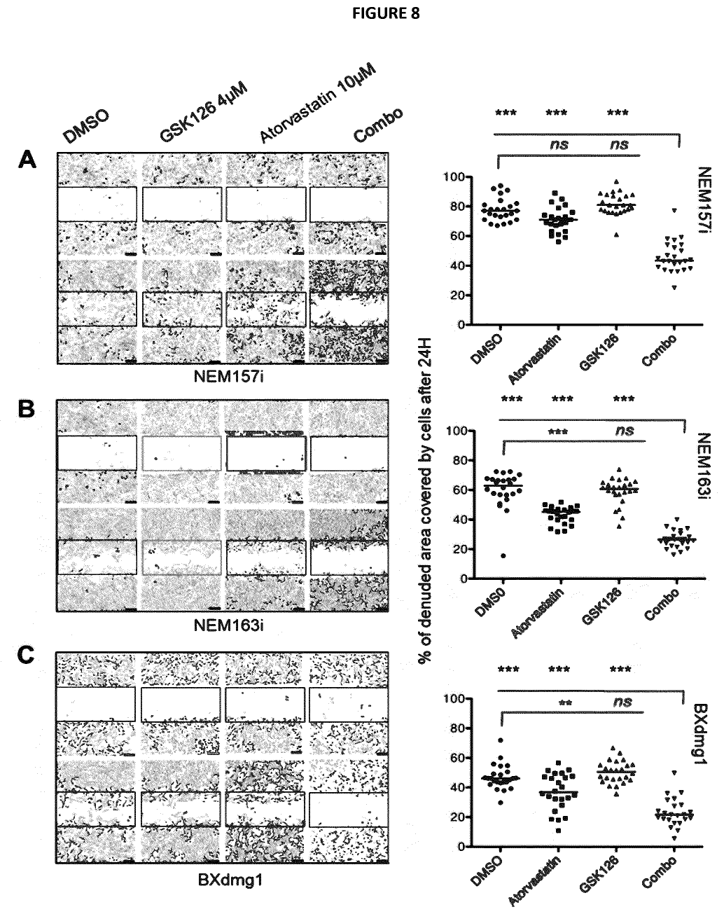

We also investigated the influence of the drugs on DMG cell migration using an automated cell scratcher with the Live-Cell IncuCyte® S3 Analysis System (Sartorius). Cell movement was measured from 24h to 48h after confluence and initiation of wound. Initial denuded area is very clean and marked by a frame of the same size for illustration in all conditions (FIG. 8A-C). Percent of denuded area covered by migrating DMG cells NEM157i, NEM163i and primary BXdmg1 cells after 24h was reported by the Incucyte software and is displayed per condition in the right graphs (FIG. 8A-C). For all cells tested, a significant inhibition of migration activity (approximately 2-times) was observed for the combo treatment (p<0.001) compared to single treatments or solvent control. In NEM163i and BXdmg1 cells, migration was also slowed by Atorvastatin compared to DMSO (p<0.05 and p<0.001), but cells exposed to combo treatment still had fewer area covered by migrating cells compared to Atorvastatin alone (p<0.001).

### Inhibition of Tumor Cell Spheroid Formation by Atorvastatin/GSK-126 Combination

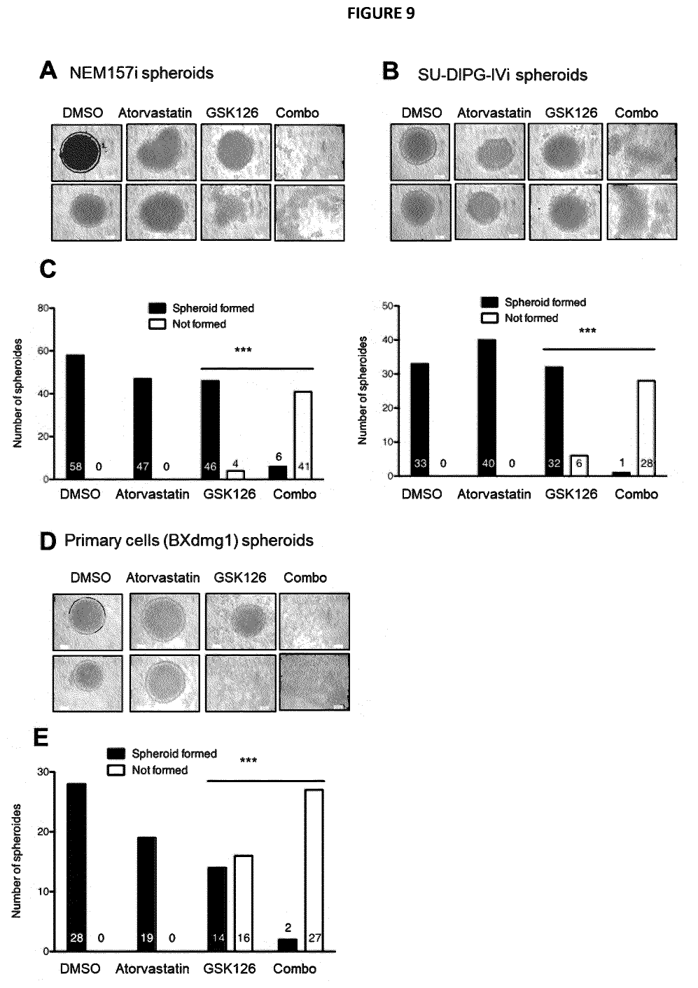

In an attempt to further investigate cell movements and adhesion phenomena in our cells, we used a protocol which allows spheroid formation of DMG cells and analyzed cell movements and aggregation with an IncuCyte S3 Analysis System. Tumor spheroids are considered as a more realistic culture system than classical 2D models, adding 3D complexity closer to in vivo growth conditions. Using three different DMG cell lines, including our primary BXdmg1 cells, we could reliably generate tumor neurospheres with all cell lines (FIGS. 9A, 9B, 9D). Round-shaped spheres formed rapidly within 24h, albeit with slightly different sizes and sometimes differences in border shape (clear delimitated vs. irregular). However, cells exposed to combo-treatment almost never formed spheroids compared to single GSK-126 treatment (p<0.001, for all cells, FIGS. 9C and 9E). In some GSK-126-treated cultures, spheroid formation was affected, especially in the primary cells (FIGS. 9D, 9E).

### GSK-126 Effect on Orthotopic SU-DIPG-IVi-Luc Implanted Tumor Cells in NOD/LtSz-Scid IL.2Rgamma (NSG) Mice.

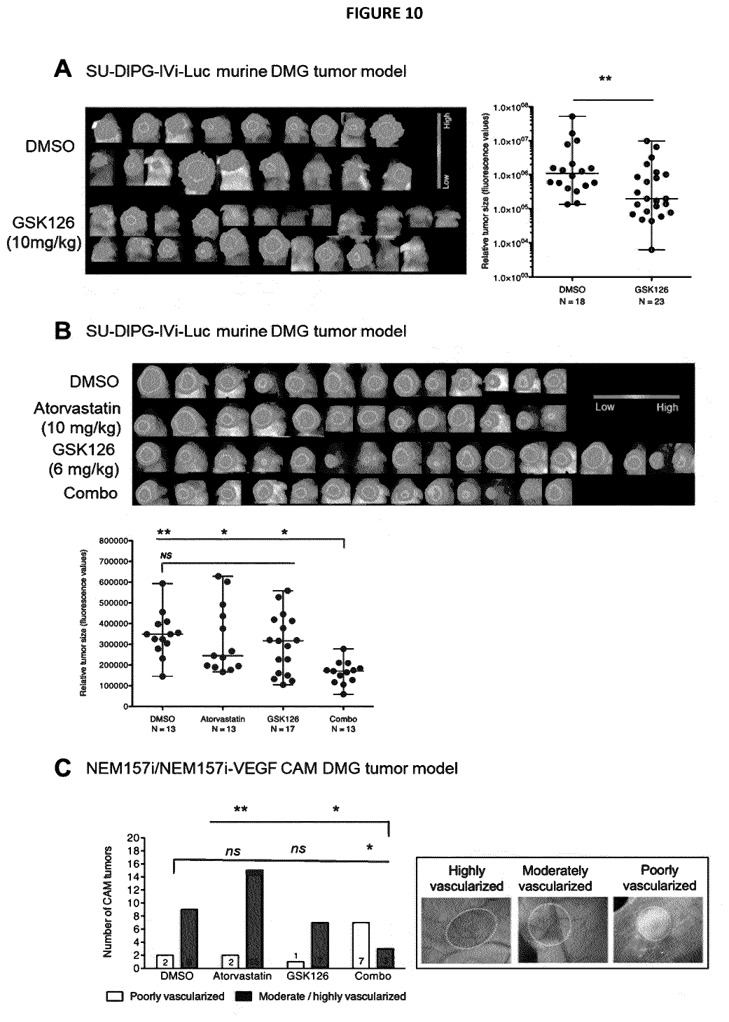

We developed an ortothopic brainstem glioma model in newborn immunocompromised mice. SU-DIPG-IVi-Luc cells where generated using lentiviral transfection procedures. In a first experiment, we tested efficacy of GSK-126 treatment in this model and found significant growth reduction at an i.p. dose of 10 mg/kg evidenced by bioluminescence (p=0.009) in treated mice (n=23) compared to solvent controls (n=18, FIG. 10A). For the Atorvastatin/GSK-126 combo treatment experiment, we reduced GSK-126 dose to 6 mg/kg to avoid growth inhibition. After four treatments, a significantly greater tumor growth inhibition of the combo occurred (n=13) compared to Atorvastatin (n=13), GSK-126 (n=17) (p<0.05) as well as to DMSO controls (p<0.01, n=13, FIG. 10B). No significant differences have been found between controls or single treatments. All bioluminescence images of animals used in this study are shown (FIG. 10A, B). Atorvastatin/GSK-126 combo treatment also exhibits better anti-tumor effects in a chick CAM DMG model we recently developed. In this short-term model, based on a previously established adult glioma CAM model, drugs can be directly applied on the tumor and growth monitored by biomicroscopy. Phenotypic characterization of drug action can be made by classifying the degree of tumor vascularization into high or low/moderate (FIG. 10C, right panel). Degree of vascularization should be interpreted as capacity of tumor cells to interact with the host tissue, an indirect indicator of tumor cell aggressiveness. Combo treated experimental tumors showed reduced vascularization compared to Atorvastatin (p=0.036), GSK-126 (p=0.0248) and DMSO controls (p=0.03, FIG. 10C, left graph).

### Example 2: Tri-Therapies Efficiency for Treating and/or Preventing Hepatoblastoma EZH2 is a Central Oncogene in Proliferative Hepatoblastoma

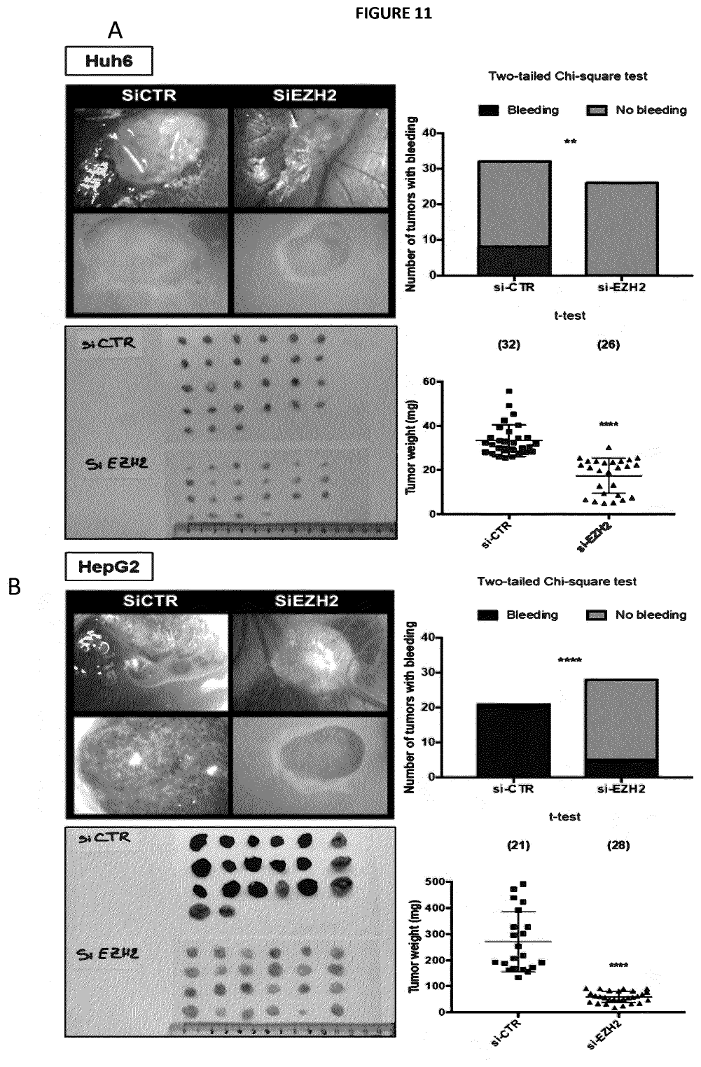

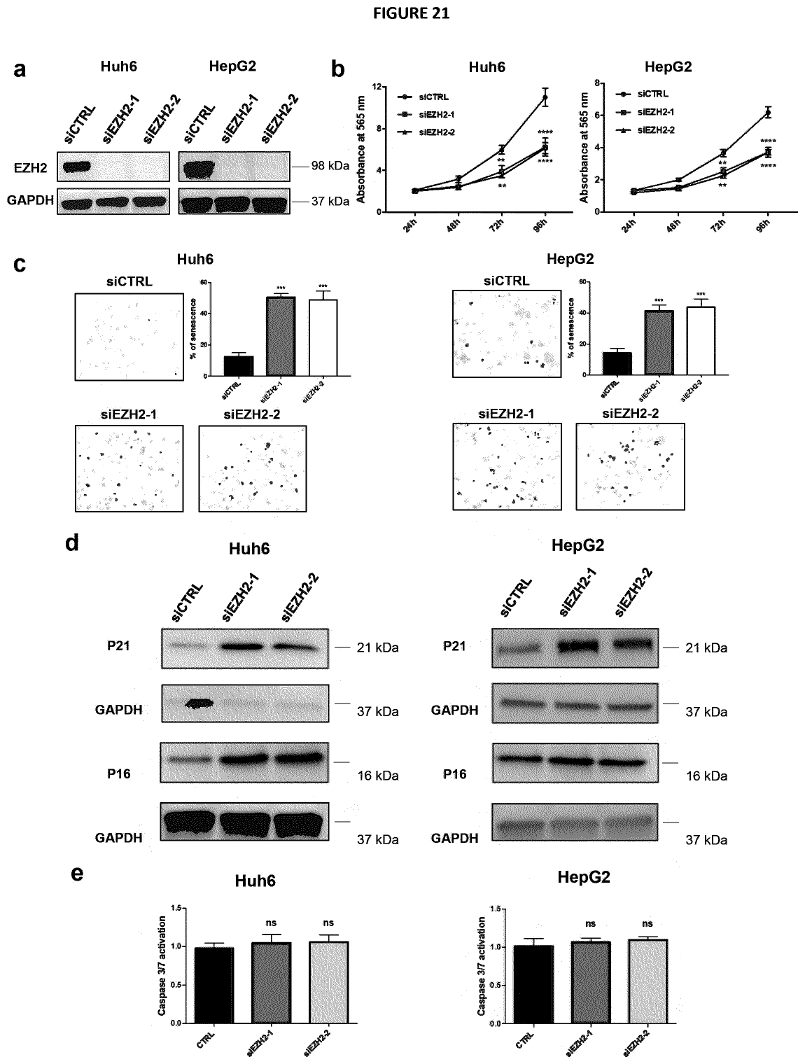

Using classical 2D culture and 3D spheroid models, we found that the depletion of EZH2 by RNA interference blocks the growth of the HB-deriving cell lines Huh6 and HepG2 in vitro by activating senescence (see Example 4 below and FIG. 21). In vivo, the depletion of EZH2 totally impeded the development of hepatoblastoma using the chorioallantoic membrane assay (CAM, Indersie et al, Oncotarget, 2017, PMID: 29662633) and reduced tumor cell aggressiveness by lowering tumor angiogenesis (FIG. 11). Therefore, EZH2 is a relevant therapeutic target for the treatment of hepatoblastoma.

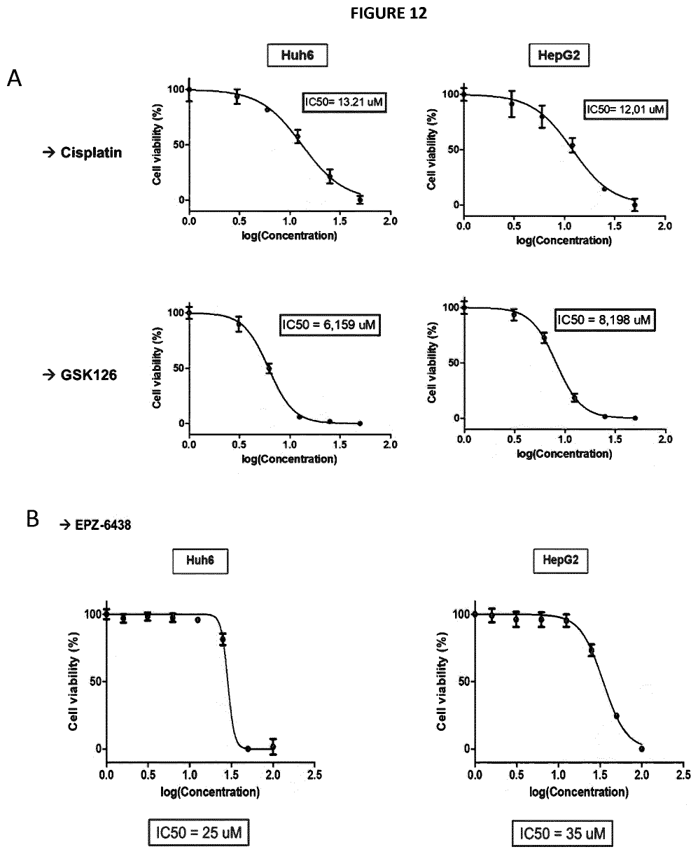

We then measured the effect of GSK-126 and Tazemetostat (also known as EPZ-6438-see https://pubchem.ncbi.nlm.nih.gov/compound/66558664), two EZH2 inhibitors, on the growth of hepatoblastoma cells. While Tazemetostat was effective at concentrations above 20 μM (FIG. 12), the IC50 (half maximal effective concentration) of GSK-126 was around 6 to 8 μM, a concentration twice lower than that of cisplatin (FIG. 12).

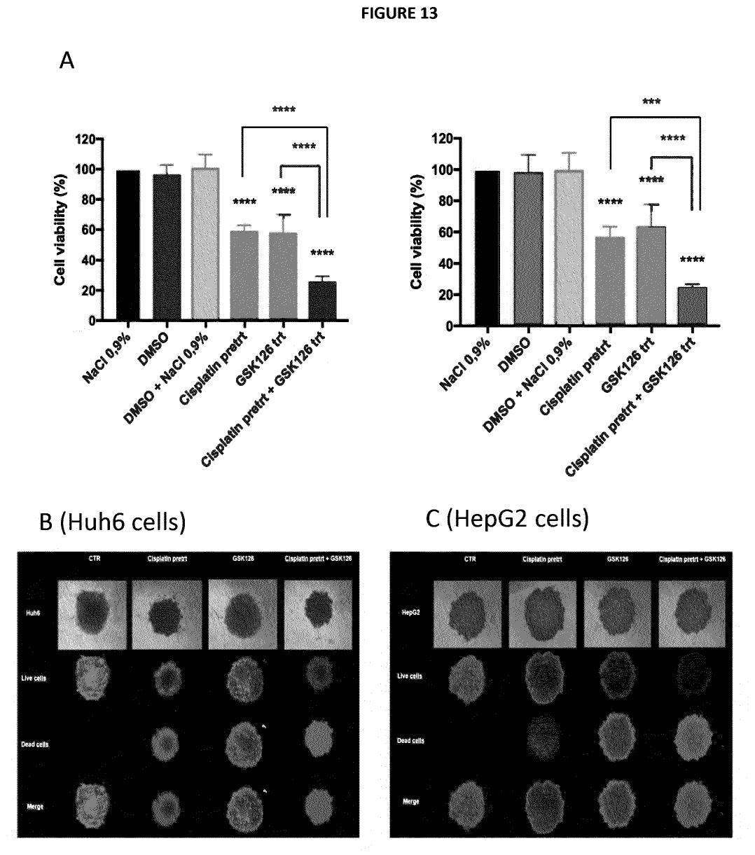

Thus, we tested the effect of combining cisplatin and GSK-126 (see IC50s measured for each drug in each cell line in FIG. 12A) to eliminate hepatoblastoma cells using classical 2D culture condition and 3D tumor spheroid. As shown in FIG. 13, GSK-126 and cisplatin had an additive effect to eliminate hepatoblastoma cells in both culture models.

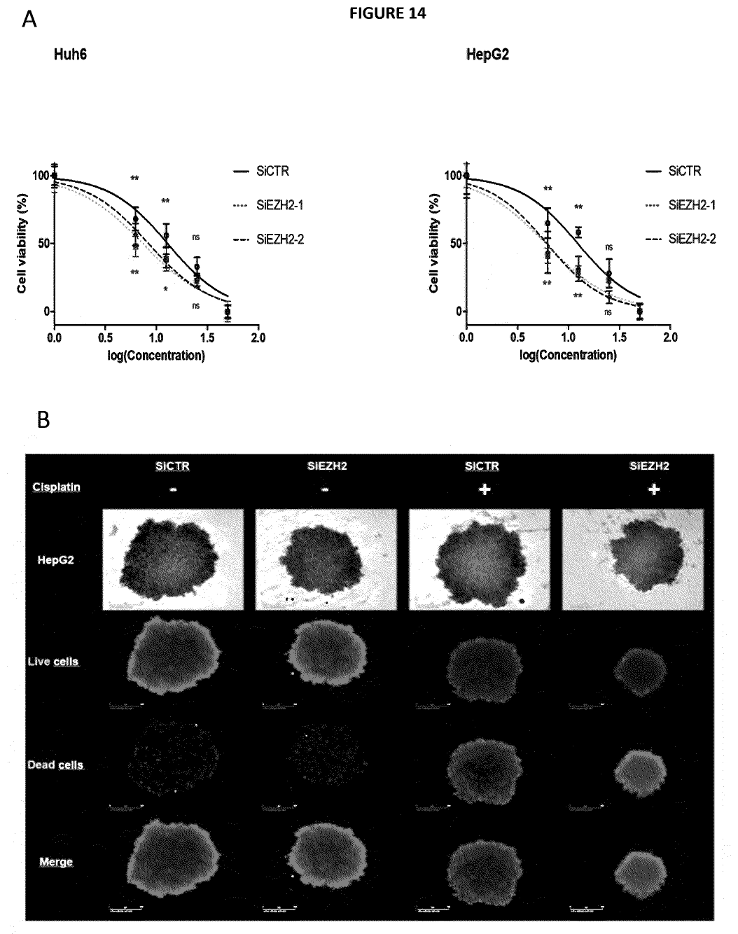

Subsequently, we tested the hypothesis that EZH2 participates in the resistance of hepatoblastoma cells to cisplatin, a situation often observed in relapsed patients treated with cisplatin. As shown in FIG. 14A, the sensitivity of Huh6 and HepG2 cells to cisplatin was increased by 35 to 48% in absence of EZH2 protein demonstrating the participation of EZH2 in cisplatin resistance. cisplatin was also more potent to eliminate hepatoblastoma cells cultured as 3D spheroids when they do not express EZH2 (FIG. 14B).

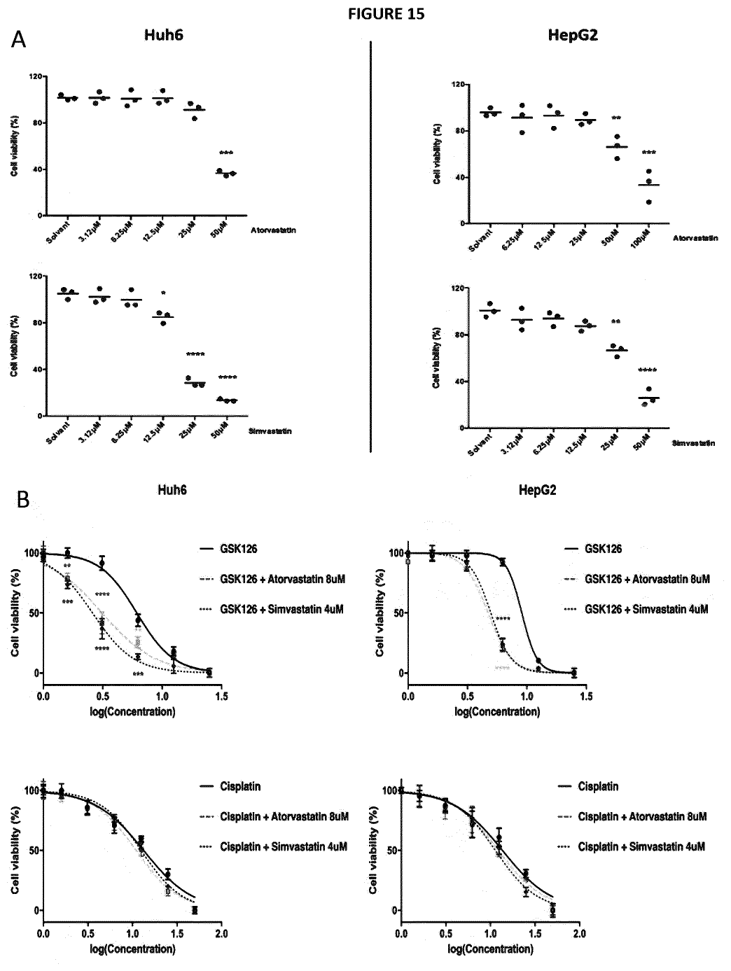

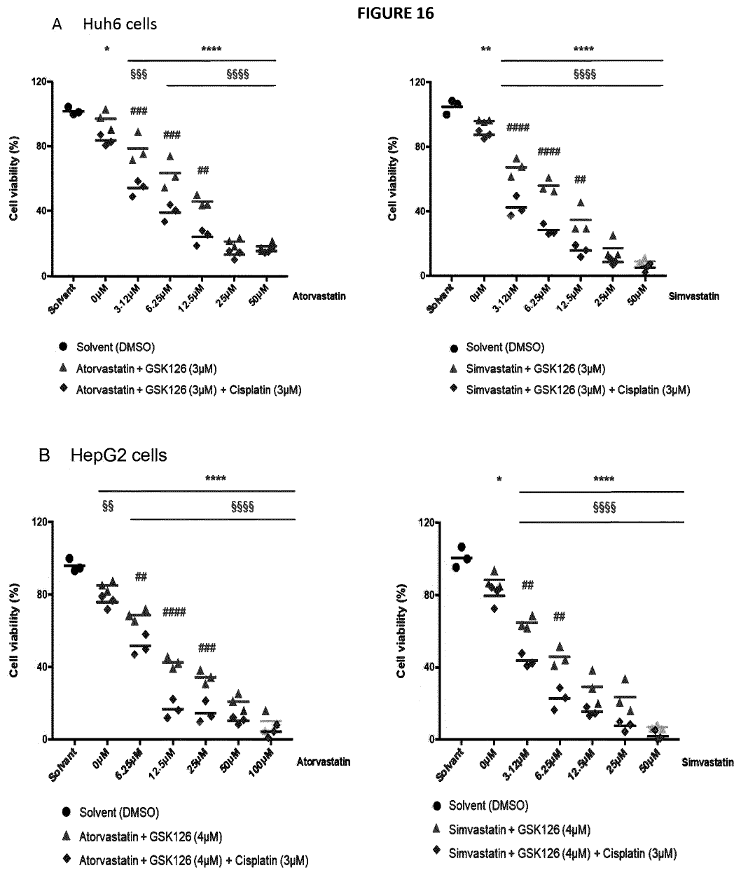

As the EZH2 inhibitor GSK-126 efficiently eliminates hepatoblastoma cells and deregulates cholesterol metabolism in DMG cells (see Example 1.2), we tested whether statins synergize with GSK-126 and/or cisplatin to eliminate these tumoral hepatic cells in vitro. As shown in FIG. 15A, Simvastatin or Atorvastatin alone had an antitumor effect on Huh6 and HepG2 cells at a concentration of 20 μM and 50 μM and above, respectively. As expected, the sensitivity of Huh6 and HepG2 cells to GSK-126, but not to cisplatin, was significantly increased (at least twice) in presence of Simvastatin or Atorvastatin (FIG. 15B) showing that statins synergize with GSK-126 to eliminate hepatoblastoma cells, but not with cisplatin. Next, we tested a tri-therapy by combining GSK-126, cisplatin and statins. As shown in FIG. 16A-B, the sensitivity of Huh6 and HepG2 cells to the combination of GSK-126+cisplatin (3 μM each) was significantly increased in presence of Simvastatin or Atorvastatin. The tri-therapy GSK-126+cisplatin+statin was significantly more efficient than the combination GSK-126+Statin to kill hepatoblastoma cells (FIGS. 16A-B). Again, Simvastatin was slightly more efficient than Atorvastatin to potentiate the antitumor effect of GSK-126 alone or in combination with cisplatin.

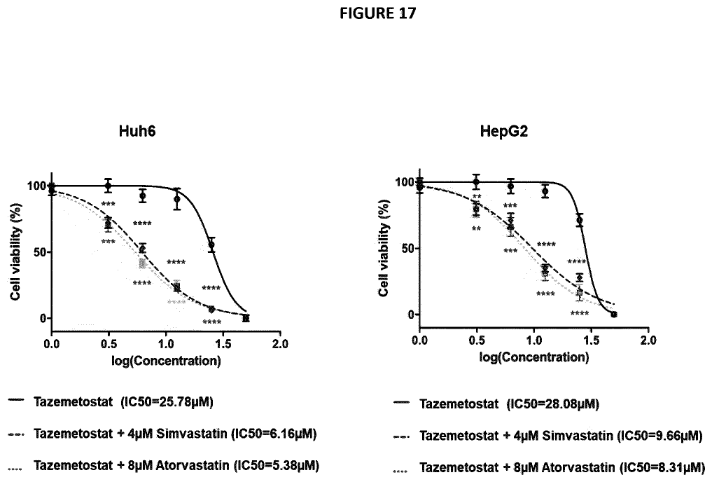

Finally, we tested the hypothesis that the synergistic effect of GSK-126 and statins could be extended to other EZH2 methyl-transferase inhibitors. Remarkably, data in FIG. 17 showed that Simvastatin or Atorvastatin significantly potentiates the sensitivity of hepatoblastoma cells to the EZH2 inhibitor Tazemetostat (E7438/EPZ6438) supporting our findings that statins likely synergize all EZH2 inhibitors.

Altogether, these in vitro and in vivo data in DMG and hepatoblastoma cells clearly showed that the inhibition of EZH2 enzymatic activity by EZH2 inhibitors (GSK-126, Tazemetostat . . . ) activates cholesterol biosynthesis, an adaptive metabolic process which helps tumoral cell to survive and resist to the deleterious effects mediated by EZH2 inhibitors. By combining statins and EZH2 inhibitors, tumoral cells cannot synthesis cholesterol mediated by EZH2 inactivation and are programmed for death through apoptotic processes.

### Example 3: EZH2 Upregulation is Associated with Proliferative Hepatoblastoma and Poor Prognosis Indicators

### Example 3.1: Materials and Methods

### Cell Culture

The human HB cell lines Huh6 and HepG2 were cultured as monolayer in DMEM 1 g/L and DMEM 4.5 g/L respectively (Dulbecco's modified Eagle's medium, Gibco). The culture media was supplemented by 10% fetal bovine serum (FBS, Sigma) and penicillin/streptomycin (1,000 units/mL) (Gibco). All cells used were regularly authenticated using short tandem repeat (STR) profiling and tested for mycoplasma infection twice a month.

For 3D culture, spheroids were formed with 10,000 cells per well in low attachment 96-well plate. In each well, the cells were mixed with 100 μl of media and 100 μl of methylcellulose at final concentration of 0.5%. Plates with spheroids were incubated and regularly scanned using an IncuCyte® S3 live cell analysis system (Essen BioScience).

siRNA Transfection

Cells were transfected with two different siRNAs targeting EZH2 mRNA [siEZH2-1 as set forth in SEQ ID NO: 11: GAG GGA AAG UGU AUG AUA A (TT); siEZH2-2 as set forth in SEQ ID NO12: UUU GGC UUC AUC UUU AUU G (TT)] or a control RNA (AllStars Negative Control siRNA, Qiagen). The transfection was carried out in 6-well microplates with 250,000 cells per well. siRNAs were diluted in 1×siMAX dilution buffer (6 mM HEPES, 20 mM KCl, 0.2 mM MgCl2, pH=7.3; Eurofins, Ebersberg, Germany). siRNAs were brought into contact with lipofectamine (RNAiMax Invitrogen) diluted to 1/100 in the transfection medium (OptiMEM, Gibco). A final concentration of 20 nM was used for each siRNA. The mixture was incubated for 20 minutes at room temperature to allow the formation of liposomes and then added to the cells incubated in a medium without antibiotic for 6 hours. After transfection, the medium was replaced with fresh medium with antibiotics.

### Mutagenesis and Plasmid Construction

To carry out the cloning, the lentiviral plasmid pSIN-EF1alphaL-eGFP-IRES-Puro was used. Two inserts corresponding to wild-type EZH2 and mutated version of EZH2 (EZH2*) were cloned. Briefly, wild-type EZH2 open reading frame was amplified by PCR using cDNAs ordered from SinoBiological as matrix and the following primers: Forward: 5′-GCG CGC TAG CAC CAT GGG CCA GAC TGG GAA-3′ as set forth in SEQ ID NO: 13; Reverse: 5′-GCG CAC GCG TTC AAG GGA TTT CCA TTT CTC-3′ as set forth in SEQ ID NO: 14. H689A mutant referred to as EZH2* was obtain by mutagenesis as described by Jung Kim et al. (Polycomb- and Methylation-Independent Roles of EZH2 as a Transcription Activator. Cell Rep, 2018. 25 (10): p. 2808-2820 e4) using the following primers: Forward: 5′-GTTTGGATTTACCGAAGCATTTGCAAAACGAATTTTGTTACCCTTGCG-3′ as set forth in SEQ ID NO: 15 and Reverse: 5-CGCAAGGGTAACAAAATTCGTTTTGCAAATGCTTCGGTAAATCCAAAC-3′ as set forth in SEQ ID NO: 16. The PCR product was cloned in the NheI/MluI sites of pSIN-EF1αL-eGFP-IRES-Puro vector and fully sequenced before being sent to the Vectorology core facility for lentivirus production.

### Lentiviral Production, Titration and Cell Transduction

Production and titration of infectious lentiviral particles was done by Vectorology platform VECT'UB. Procedures and policies have been described previously by Maurel M et al (A functional screening identifies five microRNAs controlling glypican-3: role of miR-1271 down-regulation in hepatocellular carcinoma. Hepatology, 2013. 57 (1): p. 195-204), and Laloo B et al (Analysis of post-transcriptional regulations by a functional, integrated, and quantitative method. Mol Cell Proteomics, 2009. 8 (8): p. 1777-88). The lentiviral particles were added to the cells and incubated for 24 hours. After that, the cells were washed twice with PBS and then cultured for a few days before experimental use. The ectopic expression of proteins was confirmed for each experiment.

### Drug Treatments

cisplatin (S1166), EPZ6438 (also known as Tazemetostat, S7128), GSK-126 (S7061), Atrovastatin (S5715) and Simvastatin (S1792) were purchased from SelleckChem. cisplatin was dissolved with NaCl 0.9%. EPZ6438, GSK-126, Atrovastatin and Simvastatin were dissolved with DMSO (dimethyl sulfoxide). For Simvastatin, the drug was activated by NaOH in EtOH treatment prior to use in cell assays. All drugs were stored at −20° C.

### Cell Growth and Viability Assay

For 2D culture, a total of 2,000 cells/well for Huh6 and 3,000 cells/well for HepG2 were seeded in 96-well microtiter plates. Cell growth was measured at 24 h, 48h, 72h or 96h after genetic manipulation and at 48h or 72h after drug treatment using the in vitro MTS assay Kit or Sulforhodamine B (SRB, Absorbance at 565 nm) assay (Sigma) following manufacturer's instructions. For 3D culture, spheroids were formed as described above and microplates were incubated in an IncuCyte® S3 live cell analysis system and scanned every 8 hours. For genetic manipulation, the lentiviral transductions or the gene depletions were done before the plating. For drug treatments, depending on the experiment and the drug, spheroids were treated at day 4 or 5 for 48h or 72h. Cell viability assay in spheroids was done using 1 μM calcein AM (BioLegend) and 2 μM ethidium homodimer 1 (Sigma) for 30 minutes at 37° C. Spheroids were imaged using an IncuCyte® S3 live cell analysis system. For all cell viability assays, drugs were used at half maximal inhibitory concentration (IC₅₀).

### Cell Senescence

EZH2-silenced or control cells were seeded into a 24 well plate. Three days later, cells were fixed and beta-galactosidase activity was measured by Senescence assay using the beta-Galactosidase Staining kit (Cell Signaling, Danvers, Massachusetts, USA) according to manufacturer's instructions. Cells were observed and imaged using an InCellis microscope (Bertin Technologies, France). Senescent cells were counted using ImageJ.

### Apoptosis

Apoptosis was measured in EZH2-silenced or control cells seeded into a 96 well plate after genetic manipulation or drug treatment using caspase 3/7 activity assay (Promega Corp., Madison, WI, USA).

### Cell Migration

EZH2-silenced or control cells were plated into the 2-well cell culture inserts (Biovalley, France) at a density of 35,000 cells per well. Eighteen hours later, cell migration was imaged at 0, 8 and 24 hours with an InCellis microscope (Bertin Technologies, France). Quantification was performed with Image J.

For the study of drugs effect on cell migration, cells were plated in a 96-well plate at a density of 50,000 cells per well and incubated until attachment. An IncuCyte WoundMaker (96-pin woundmaking tool) was used to make scratches. Then, cells were treated with drugs at IC₂₅ and cell migration was monitored by scanning each well every hour for 24 hours using an IncuCyte® S3 live cell analysis system.

### Lipid Droplets Assay

The cells were plated in 6-well plate at a density of 250,000 cells per well. 24 hours later, the cells were treated or not with the drug at the previously determined IC₅₀. After 48 hours of treatment, the cells were fixed in 4% PFA for 15 minutes then stained with the red oil for 15 minutes at room temperature. Finally, cells were washed several times and scanned using InCellis microscope (Bertin Technologies, France).

### Western Blots

Cells were lysed and total proteins were extracted 48 hours after treatment or 72 hours after genetic manipulation using a mixture of RIPA buffer (Sigma), protease inhibitors and phosphatase inhibitor cocktail (Roche). After protein quantification with BCA Protein Assays (ThermoFisher), 40 μg of total proteins were loaded in 4-15% pre-casted gels (Bio-Rad) for migration. Then, proteins were transferred onto a nitrocellulose membrane (Transblot® Turbo midi-size, Bio Rad). The membranes were blocked with 5% BSA or Odyssey blocking buffer (LI-COR Biosciences) and detected with the corresponding antibodies (Table 2). The revelation was performed by chemiluminescence using Fusion FX (Vilber Lourmat) and quantification was done using ImageJ software (National Institutes of Health, Bethesda, Maryland, USA).

TABLE 2
Antibodies used for western blot analyses.

| Target protein | Supplier | Cat no. | Dilution |
|---|---|---|---|
| EZH2 | Cell Signaling | 5246S | 1/1000 |
| P16 | Abcam | Ab108349 | 1/1000 |
| P21 | Cell Signaling | 2947 | 1/1000 |
| P44/42 MAPK(ERK1/2) | Cell Signaling | 9102 | 1/1000 |
| Phospho-p44/42 (p-ERK1/2) | Cell Signaling | 9106 | 1/1000 |
| HMGCR | Atlas Antibodies | CL0259 | 1/1000 |
| H3K27me3 | Ozyme | 9733S | 1/1000 |
| Histone H3 | Santa-Cruz | 517576 | 1/500 |
| GAPDH | Ozyme | BLE649203 | 1/15000 |
| Anti-mouse IgG-HRP | Biorad | 170-6516 | 1/3000 |
| anti-rabbit IgG-HRP | Sigma Aldrich | A0545 | 1/5000 |

### Proteomics

Proteomic analysis was performed, in Huh6 cell line depleted EZH2 or control, by the Proteomics Core Facility at the University of Bordeaux (https://proteome.cgfb.u-bordeaux.fr/en). All steps were done as described by Ghousein et al (miR-4510 blocks hepatocellular carcinoma development through RAFI targeting and RAS RAF MEK ERK signalling inactivation. Liver Int, 2020. 40 (1): p. 240-251).

### Chick CAM Assay

Animal procedures were carried out as described before (Indersie, E., et al., Tracking cellular and molecular changes in a species-specific manner during experimental tumor progression in vivo. Oncotarget, 2018. 9 (22): p. 16149-16162) (Indersie, E., et al., MicroRNA therapy inhibits hepatoblastoma growth in vivo by targeting beta-catenin and Wnt signaling. Hepatol Commun, 2017. 1 (2): p. 168-183) in agreement with the European (directive 2010/63/UE) and French (decree 2013-118) guidelines. Briefly, Fertilized embryos were received at the stage of segmentation. Then, they were incubated at 37.4° C. and 70% humidity. At day 3 of development, the eggshell was opened on the top and the opening sealed with medical-grade Durapore tape. At day 10 of embryonic development, 1 million Huh6 or HepG2 cells were embedded in Matrigel® (growth-factor reduced, Corning) droplets (40 μL) and deposited on the CAM. Tumor growth and vascularization were monitored by stereomicroscopy (SMZ745T) and pictures were taken using camera (DS-Fi2) on day 1, 3 and 7. At day 7, all tumors were fixed with PFA 4% and extracted. Then the weigh was measured using a precision balance.

### Clonogenicity Assay

Huh6 and HepG2 cells were seeded at 500 and 1000 cells/well respectively in 12-well plates. After attachment, cells were treated with drugs. For Huh6, Atorvastatin (8 μM), Simvastatin (4 μM) and/or GSK-126 (3 μM) and for HepG2, Atorvastatin (8 μM), Simvastatin (4 μM) and/or GSK-126 (4 μM).

Following incubation at 37° C. for 10 days, the cells were fixed with 4% PFA and stained with 0.05% crystal violet. Reading under the Fusion FX (Vilber Lourmat).

### Toxicological Assay in Xenopus Embryos

Batches of gastrula stage embryos (10 embryos by batch) were incubated in 24-well plates in presence of cisplatine or GSK-126 at the indicated concentrations. Embryos were left in solution until untreated control embryos reached stage 41. Control or cisplatine-treated embryos were incubated in 0.1× Marc's Modified Ringer. GSK-126-treated embryos were incubated in 0.1× Marc's Modified Ringer supplemented with 0.1% DMSO.

### Tumor Xenografts

NOD/LtSz-scid IL2R gamma null (NSG) mice were bred in standard conditions compliant with regulatory bodies (French government). Sterilized food and water were accessible ad libitum. One million of Huh6 cells in 50% of Matrigel were subcutaneously injected in a total volume of 100 μl in the right flank of 8- to 9-week-old female mice (26-32 g; APAFIS #32917-2021121316283534 v2, Agreement #B33063916, Ministère de l'Enseignement Supérieur, de la Recherche et de l'Innovation).

Tumor growth was monitored by caliper measurement until tumors reached an average volume of 250 mm3 (at day 12, see arrow). Thus, mice were randomly divided in 5 groups and treated by the different drugs through intraperitoneal route. cisplatin was injected twice a week at a dose of 1 mg/kg. GSK-126 and atorvastatin (ATR) were injected 3-times a week at a dose of 50 and 20 mg/kg, respectively. The vehicle (PBS with 5% DMSO) was injected 3-times a week. Tumor growth was monitored during 16 additional days until tumors control reached a size of 2000 mm3. Thus, all mice were euthanized. Blood was collected from all euthanatized mice and circulating ASAT, ALAT, creatinine, and urea were measured to evaluate the toxicity of the different treatments on the liver and kidneys.

### Statistical Analysis

Statistical analyses were performed using GraphPad Prism 6.0 or 7.0 software. All data are displayed as a mean of at least three independent experiments and error bars indicate standard deviation (SD) of the mean. When experiment contained two unmatched groups of values, the nonparametric Mann-Whitney test was used for the comparison of means. When experiment contained two matched groups of values and depending on whether data were considered to follow a Gaussian distribution or not, the parametric t-test or the nonparametric Wilcoxon matched-pairs signed ranked test was used. When experiment contained three groups of values or more, the regular one-way analysis of variance (ANOVA). When experiment contained three groups of values or more and two experimental factors, the Two-way ANOVA was used for the comparison of multiple means and conditions. The One-way and the Two-way ANOVA tests were followed by the Sidak's multiple comparisons post-test. When experiment contained two groups of categorical variables, the two-tailed Chi-square test was used. Results were considered significant when p<0.05. For all data in figures, *: p<0.05, **: p<0.01, ***: p<0.001, ****: p<0.0001 or exact p-values where indicate.

### Transcriptomics Data

Transcriptomic data and datasets (Table 3) were as described in previous publications (Hiyama, E., Gene expression profiling in hepatoblastoma cases of the Japanese Study Group for Pediatric Liver Tumors-2 (JPLT-2) trial. 2019, Science Repository OU; Carrillo-Reixach, J., et al., Epigenetic footprint enables molecular risk stratification of hepatoblastoma with clinical implications. J Hepatol, 2020. 73 (2): p. 328-341; Sumazin, P., et al., Genomic analysis of hepatoblastoma identifies distinct molecular and prognostic subgroups. Hepatology, 2017. 65 (1): p. 104-121; Valanejad, L., et al., PARP1 activation increases expression of modified tumor suppressors and pathways underlying development of aggressive hepatoblastoma. Commun Biol, 2018. 1: p. 67; Cairo, S., et al., Hepatic stem-like phenotype and interplay of Wnt beta-catenin and Myc signaling in aggressive childhood liver cancer. Cancer Cell, 2008. 14 (6): p. 471-84; ichenmuller, M., et al., The genomic landscape of hepatoblastoma and their progenies with HCC-like features. J Hepatol, 2014. 61 (6): p. 1312-20; Hooks, K. B., et al., New insights into diagnosis and therapeutic options for proliferative hepatoblastoma. Hepatology, 2018. 68 (1): p. 89-102) or uploaded from the NCBI's Gene Expression Omnibus (https://www.ncbi.nlm.nih.gov/geo, see the accession number and reference in the corresponding graph and/or Figure legend) (Edgar, R., M. Domrachev, and A. E. Lash, Gene Expression Omnibus: NCBI gene expression and hybridization array data repository. Nucleic Acids Res, 2002. 30 (1): p. 207-10) or from the R2: Genomics analysis and visualization platform (https://r2.amc.nl).

TABLE 3

| | | | # patients (%) | # patients (%) with | |
|---|---|---|---|---|---|
| | | | with EZH2 > | HMGCR > median | |
| Source | Identifier | No of samples | median NT | NT | References |
| Buendia | / | 29 | 19 (76%) | 24 (96%) | [11] |
| et al. | | (4 NTL, 25 T) | | | |
| GEO | GSE151347 | 22 | 8 (72.72%) | 9 (81.81%) | [12] |
| | | (11 NTL, 11 T) | | | |
| GEO | GSE104766 | 44 | 16 (72.72%) | 12 (54.54%) | [13] |
| | | (22 NTL, 22 T) | | | |
| GEO | GSE131329 | 67 | 51 (96.22%) | 48 (90.56%) | [7] |
| | | (14 NTL, 53 T) | | | |
| GEO | GSE133039 | 66 | 26 (76.47%) | 22 (64.7%) | [8] |
| | | (32 NTL, 31 T, 3R) | | | |
| GEO | GSE75271 | 55 | 47 (94%) | 47 (94%) | [9]) |
| | | (5 NTL, 50 T) | | | |
| GEO | GSE81928 | 32 | 26 (89.65%) | 16 (55.17%) | [10] |
| | | (3 NTL, 29 T) | | | |

GEO: Gene Expression Omnibus;
NT: non-tumoral livers;
T: Tumors;
R: recurrence

### Example 3.2: Evidence that EZH2 is a Poor Prognosis Indicator

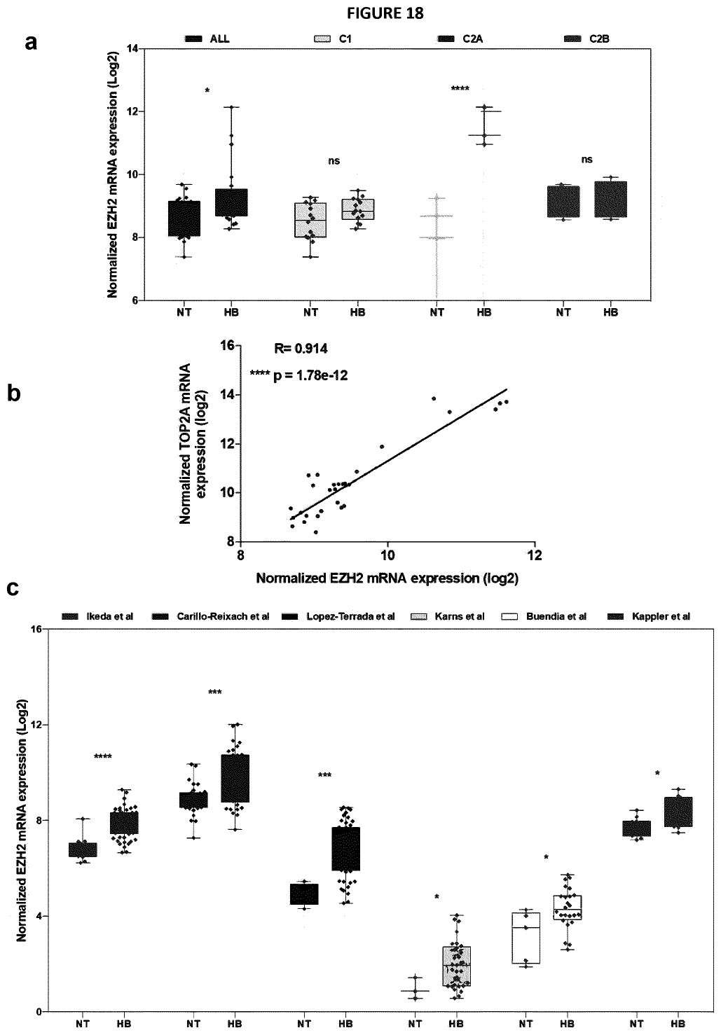

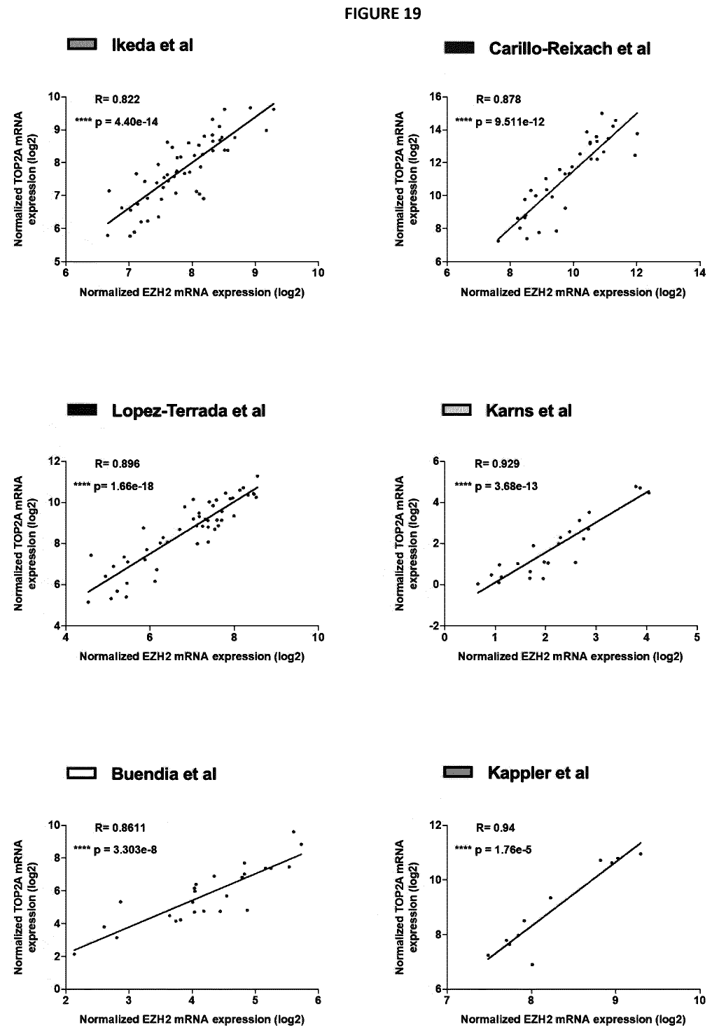

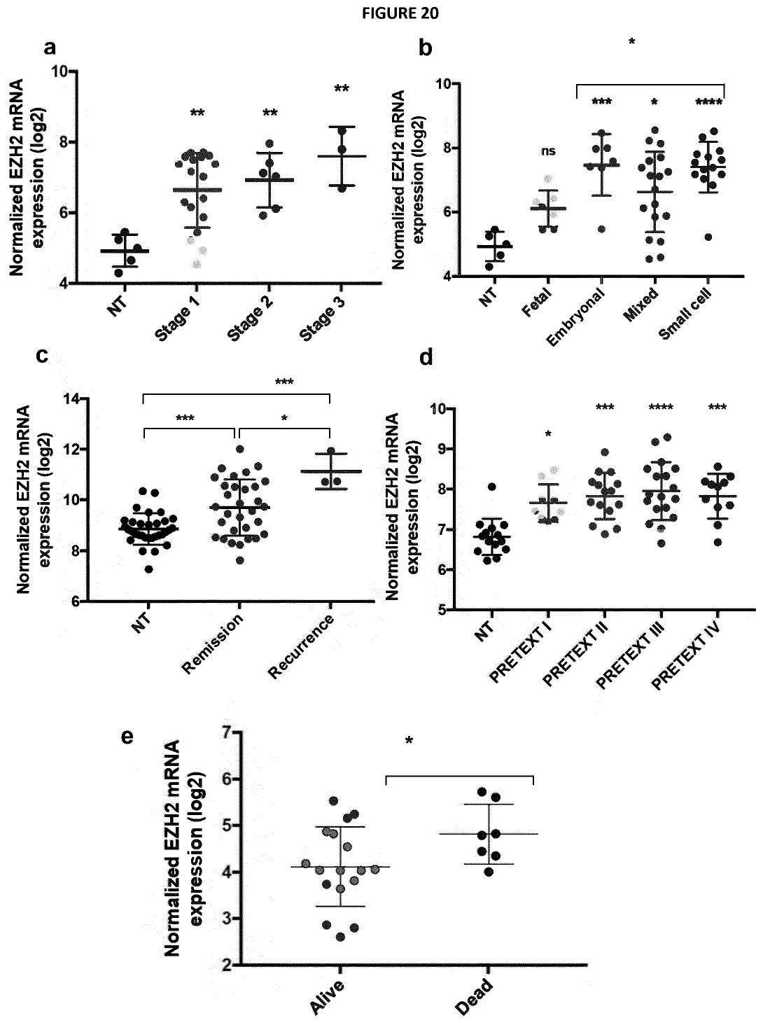

Since the role of EZH2 is poorly known in hepatoblastoma, we first analyzed the expression of EZH2 transcript in our published dataset by considering our C1, C2A and C2B tumor classification (Hooks K B et al., Hepatology, 2018 July; 68 (1): 89-102. doi: 10.1002/hep.29672). As shown in FIG. 18a, EZH2 mRNA was increased in hepatoblastoma compared to non-tumoral (NT) samples and this expression was specifically associated with the proliferative and topoisomerase 2-alpha (TOP2A) protein-positive C2A group (Hooks K B et al., Hepatology, 2018 July; 68 (1): 89-102. doi: 10.1002/hep.29672). In line with this result, a strong positive correlation was found between EZH2 and TOP2A mRNAs in hepatoblastoma samples of our cohort (FIG. 18b). To confirm these data, we analyzed EZH2 mRNA level in six additional published transcriptomic datasets. In all cases, EZH2 mRNA expression was significantly increased in hepatoblastoma (FIG. 18c), and its expression strongly correlated with TOP2A mRNA expression (FIG. 19). These data suggest that EZH2 expression could be linked to proliferative and aggressive hepatoblastoma subtypes. To confirm this trend, we performed comparative analyses using clinical and histological data. EZH2 mRNA was upregulated at all stages of tumor development as defined by Sumazin et al. (Hepatology 2017 January; 35 (1): 104-121. doi: 10.1002/hep.28888) and in all PRETEXT groups. EZH2 mRNA expression was higher in the poor-prognosis embryonal, mixed and small cell histological subtypes than in the good-prognosis fetal subtype, and it was significantly increased in recurrent tumors and in dead patients (FIG. 20). These data suggest that EZH2 could be an independent prognostic factor in hepatoblastoma.

### Example 4: EZH2 Protein Acts as a Key Oncogene in Hepatoblastoma Through its Histone Methyl Transferase Activity

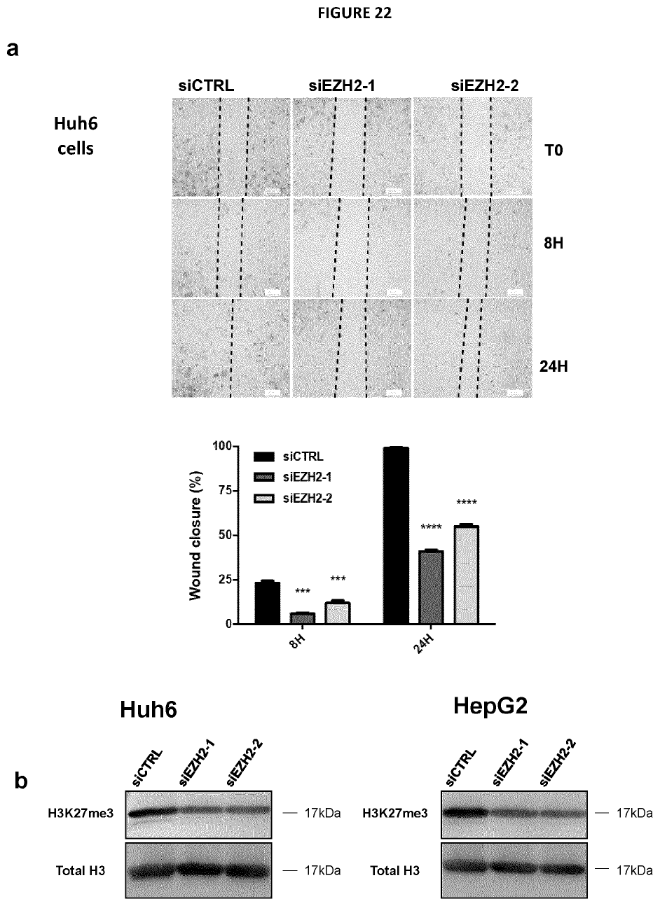

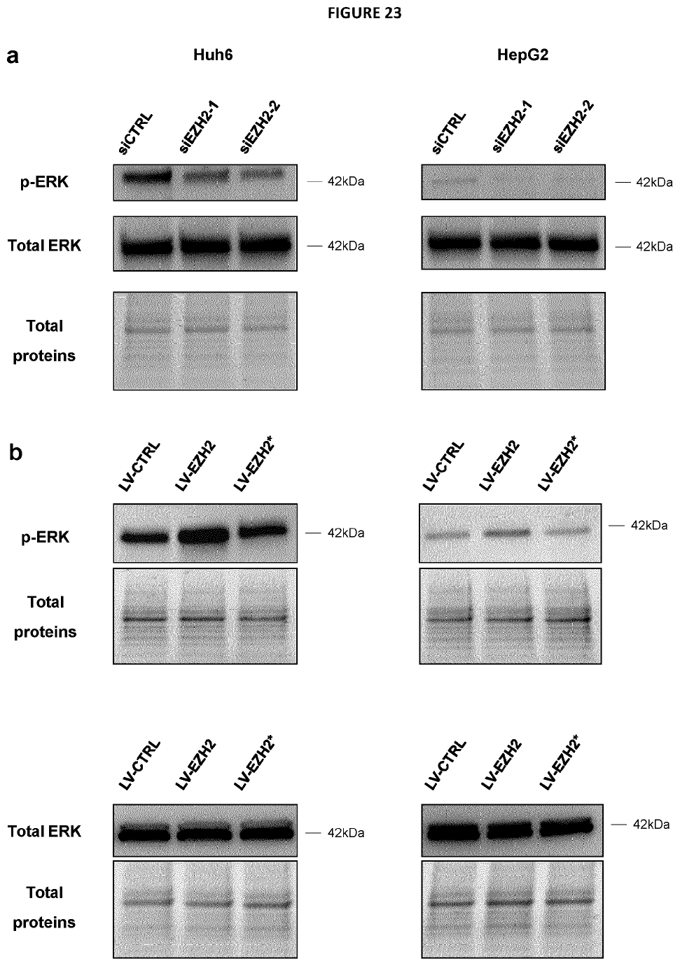

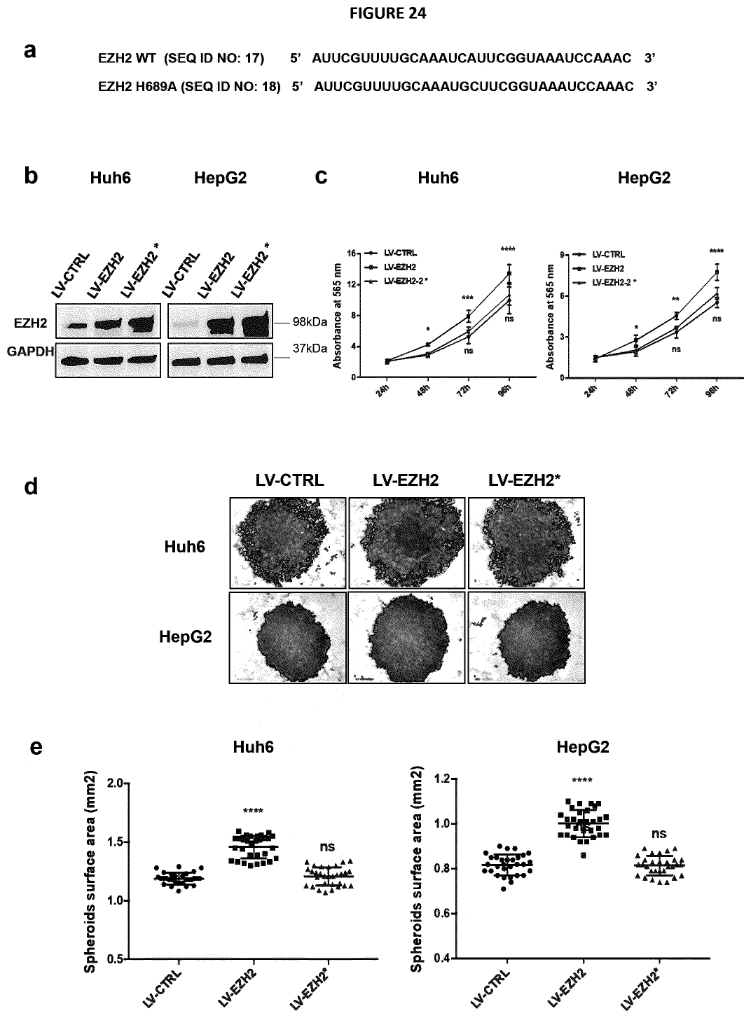

To decipher the role of EZH2 in proliferative hepatoblastoma, we first combined the use of RNA interference technology and C2A-derived hepatoblastoma cell lines Huh6 and HepG2 (Hooks K B et al., Hepatology, 2018 July; 68 (1): 89-102. doi: 10.1002/hep.29672). As shown in FIG. 21a, EZH2-1 or EZH2-2 siRNAs efficiently silenced EZH2 protein level in both cell lines 3 days after transfection. Ninety-six hours after EZH2 depletion, the growth of hepatoblastoma cells was reduced by half (FIG. 21b) and cells entered in senescence with no sign of apoptosis as illustrated by P16 and P21 increase, beta-galactosidase positive staining and the lack of caspase-3/7 activation (FIG. 21c-e). The migration of Huh6 cells was also inhibited by the loss of EZH2 protein (FIG. 22a). HepG2 cells do not migrate in our culture condition. At a molecular level, EZH2 silencing significantly reduced the trimethylation of histone H3 lysine 27 (H3K27, FIG. 22b) and the phosphorylation of extracellular signal-regulated kinase (ERK, FIG. 23a). These data suggest a relaxation of condensed chromatin in EZH2-silenced hepatoblastoma cells, the potent expression of tumor suppressors and the inactivation of the MAPK/ERK signaling, which plays a crucial role in proliferative hepatoblastoma (Mosca et al. Liver Cancer 2022; 11:126-140). To determine if the histone methyl transferase activity is essential to the oncogenic activity of EZH2 protein, we developed transgenic cell lines ectopically expressing either a wild-type EZH2 transgene or an EZH2 catalytically dead mutant, H689A (FIG. 24a, referred to as EZH2* in FIGS. 23b, 24a-e and 25). This mutant lacks the methyl transferase activity of EZH2 (FIG. 24a) (Kim et al., Cell Rep 2018; 25:2808-2820 e2804). The ectopic expression of each of these two forms of EZH2 protein was first validated by western blotting in Huh6 and HepG2 cells (FIG. 22b). Then, their specific effect was investigated in vitro. As shown in FIGS. 23b, 24c-e and 25, the wild-type EZH2 protein potentiated the phosphorylation of ERK, the growth of hepatoblastoma cells, the formation of bigger spheroids, and the resistance to cisplatin, while the mutant EZH2* protein had no effect. Altogether, these data clearly demonstrated that the histone methyl transferase activity is responsible for the oncogenic function of EZH2 in hepatoblastoma cells and their capacity to proliferate and resist to cisplatin-based chemotherapy.

### Example 5: EZH2 Histone Methyl Transferase Activity is Linked to Lipid Synthesis

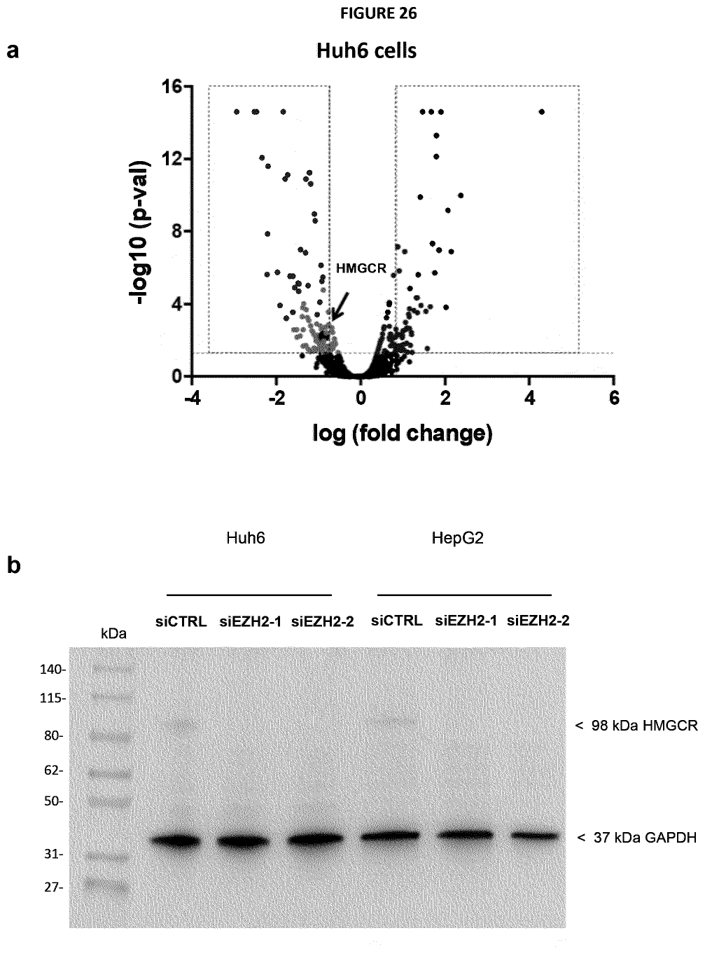

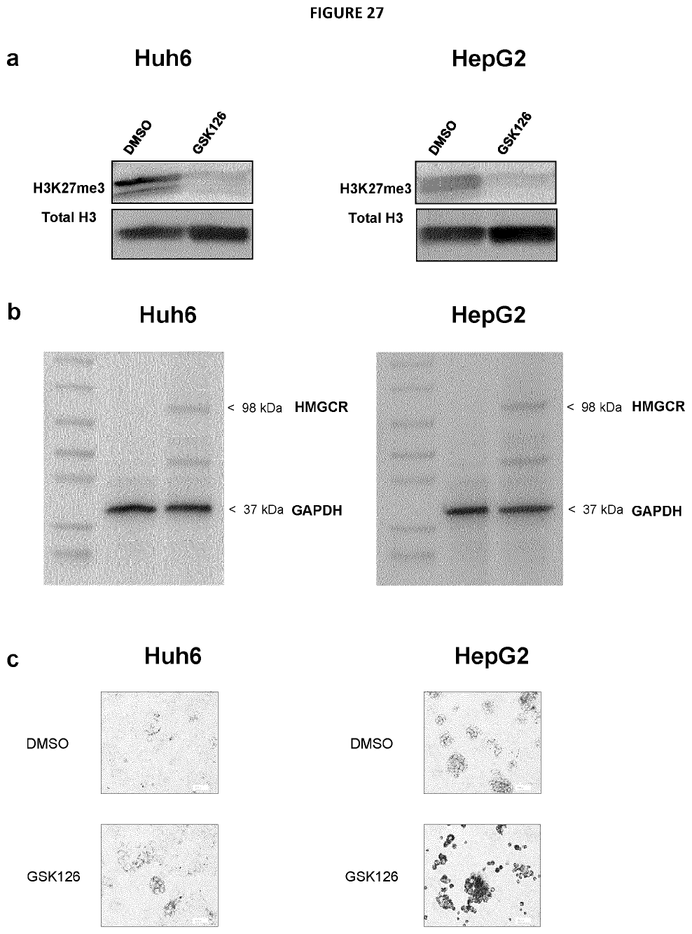

To further explore the molecular mechanisms underlying the oncogenic effect of EZH2 in hepatoblastoma we performed comparative proteomic analysis in EZH2-silenced and control Huh6 cells (FIG. 26a). Using a cut-off fold change of 1.8 and a cut-off p-value of 0.05, 54 and 87 genes were significantly and respectively up- and down-regulated in EZH2-silenced versus control cells (FIG. 26a). The up-regulation of P16 and beta-catenin proteins, as well as the downregulation of CTSV and DUSP9 proteins was assessed in Huh6 cells by western blotting and our results confirmed the accuracy of our proteomic data (FIG. 21d and data not shown). Among the down-regulated proteins, 3-hydroxy-3-methylglutaryl-CoA reductase (HMGCR) was reduced in both EZH2-silenced Huh6 and HepG2 cells (FIG. 26a-b). As expected, treatment of these cells by GSK-126, a specific inhibitor or EZH2 histone methyl transferase, reduced the trimethylation of histone H3 Lysine 27 (FIG. 27a), however, it triggered the expression HMGCR and the synthesis of lipids (FIG. 27b-c). These data suggest that, as a compensatory mechanism, hepatoblastoma cells potentiate the synthesis of lipids in response to GSK-126.

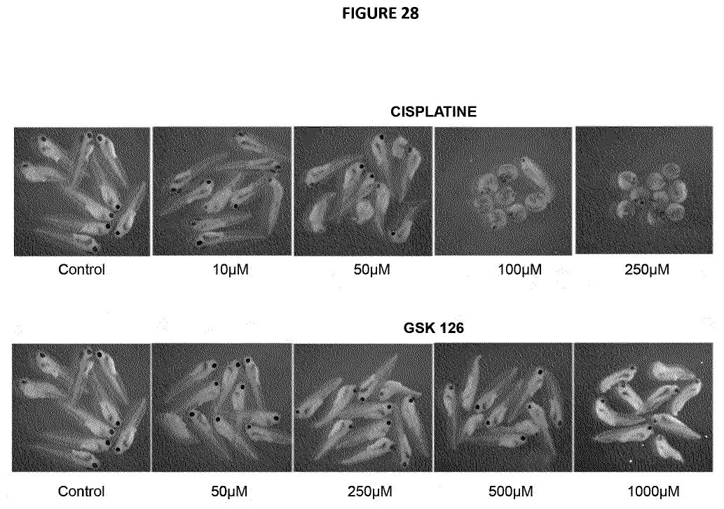

As GSK-126 efficiently blocks the activity of the oncoprotein EZH2, it could be considered as a new therapeutic option for hepatoblastoma. Therefore, we compared its toxicity to that of cisplatin in xenopus embryos. cisplatin is used for 30 years as a standard drug in the first-line treatment of hepatoblastoma. At gastrula stage, embryos were treated with increasing concentrations of cisplatin or GSK-126 (FIG. 28). Our results showed that cisplatin is toxic at a low dose of 50 μM, while GSK-126 becomes toxic at a high dose of 1 mM. Thus, GSK-126 is less toxic than cisplatin and it could be considered as a new therapeutic option in proliferative and aggressive hepatoblastoma or in the second-line therapy for patients in relapse or presenting chemoresistance to cisplatin.

### Example 6: The Combination of GSK-126 with a Statin Efficiently Eradicates Hepatoblastoma Cells In Vitro and In Vivo

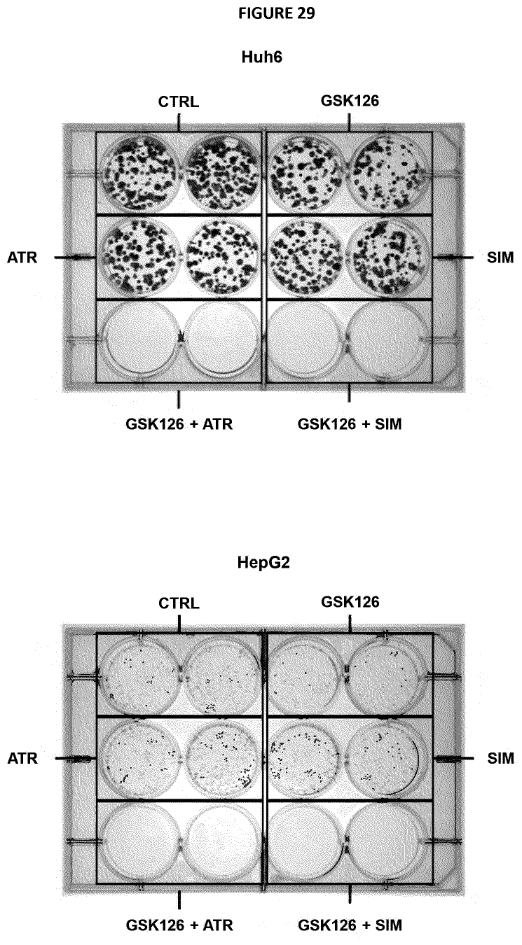

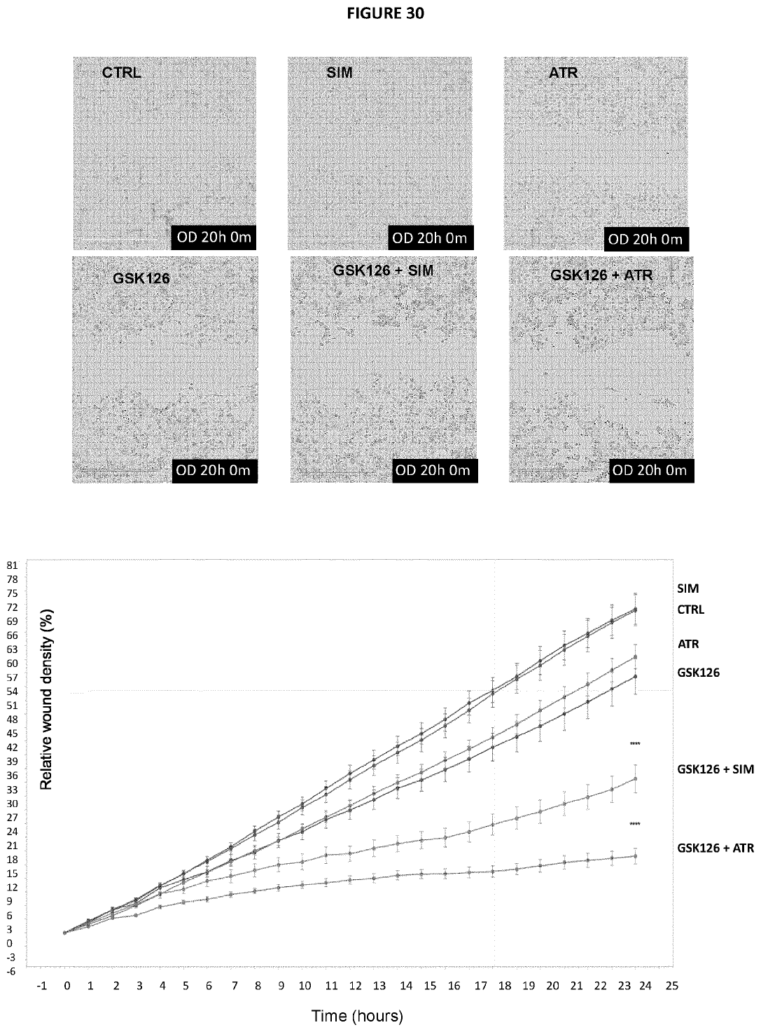

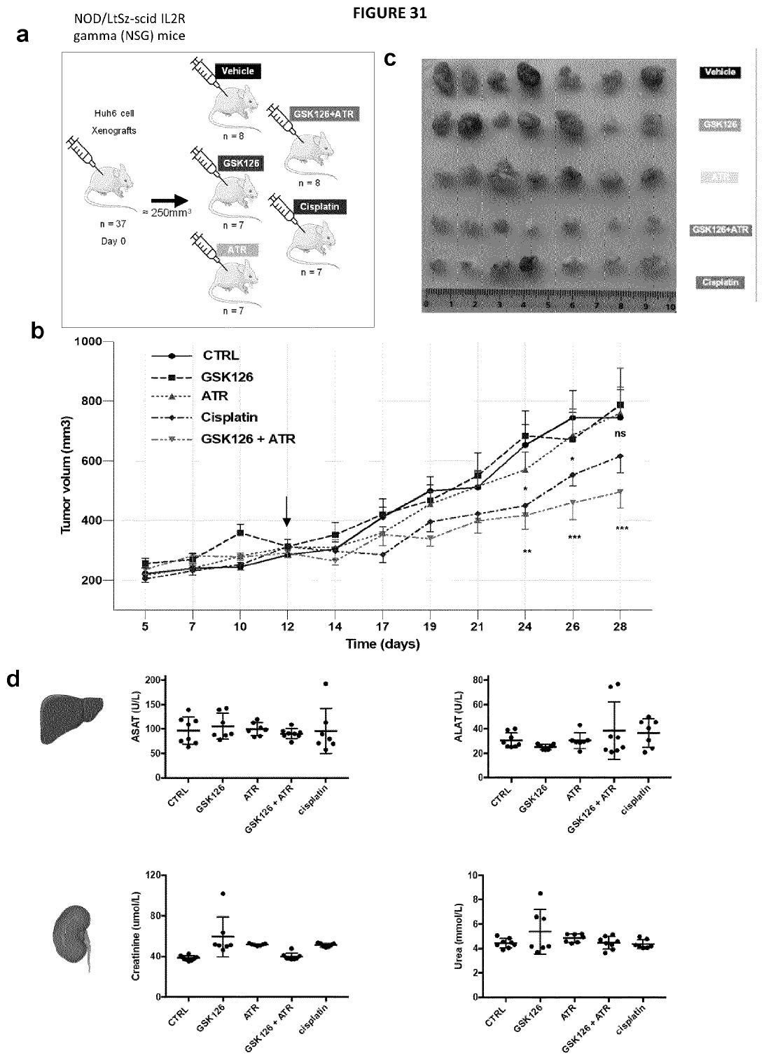

As GSK-126 induces HMGCR protein level and lipid synthesis (FIGS. 27b and c) we tested the effect of combining GSK-126 with two specific inhibitors of HMGCR enzyme, atorvastatin (ATR) or simvastatin (SIM). As shown in FIG. 29, a suboptimal dose of GSK-126 (3 and 4 μM for Huh6 and HepG2 cells, respectively), ATR (8 μM) or SIM (4 μM) alone had little effect on the proliferation and survival of hepatoblastoma cells as revealed by a clonogenic cell assay (FIG. 29). However, at the same concentrations, the combination of GSK-126 with ATR or SIM had a synergistic effect and totally eliminated hepatoblastoma cells from both cell lines in vitro (FIG. 29). In addition, the combo GSK-126+ATR or GSK-126+SIM significantly blocked Huh6 cell migration, while each drug alone had a limited effect (FIG. 30). To finish, we tested the effect of these drugs in vivo using a xenograft model of Huh6 cells in immunocompromised NSG mice (FIG. 3la) (Hooks K B, Audoux J, Fazli H, Lesjean S, Ernault T, Dugot-Senant N, et al. New insights into diagnosis and therapeutic options for proliferative hepatoblastoma. Hepatology 2018; 68:89-10). As shown in FIG. 31b, the GSK-126+ATR combination (GSK-126, 50 mg/kg; ATR, 20 mg/kg) efficiently inhibited the develop of hepatoblastoma in mice, while GSK-126 and ATR alone had no effect at the same dose. At a dose of 1 mg/kg, cisplatin also impeded tumor growth, but it was less efficient than the combination (FIGS. 11 and 31b). Following tumor extraction and by comparison with cisplatin or each drug used alone, the combo was clearly the most potent to reduce the tumor growth and vascularization (FIG. 31c). In the meanwhile, the different drugs and the combination had no effect on the blood circulating ASAT, ALAT, creatinine and urea levels demonstrating the absence of hepato- and nephrotoxicity (FIG. 31d). Altogether, our data demonstrate the benefit of combining GSK-126 with a statin to eradicate hepatoblastoma cells in vitro and blocks tumor growth and vascularization in vivo.

1. A method of treating and/or preventing tumors associated with methyltransferase EZH2, comprising administering to a subject in need thereof a therapeutically effective amount of a composition comprising a combination of an EZH2 inhibitor and one statin.

2. The method of claim 1, wherein said EZH2 inhibitor is chosen among GSK-126, UNC1999, EPZ005687, Ell, MC3629, GSK926, GSK343, GSK503, CPI-360, CPI-169, CPI-1205, tazemetostat, EPZ011989, ZLD1039, EBI-2511, pinometostat, lirametostat, JQEZ5, PROTAC MS1943, DZNep, MC1947, MC1948, and/or PF-06821497.

3. The method of claim 1, wherein tumors associated with methyltransferase EZH2 are selected among hepatoblastoma, Diffuse Intrinsic Pontine Glioma (DIPG), Diffuse Midline Glioma (DMG), bladder cancer, bone cancer, brain cancers, breast cancer, malignant lymphoma, rhabdoid tumor, leukemia, lung cancer, stomach cancer, prostate cancer, colorectal cancer, esophageal cancer, ovarian cancer, uterine cancer, liver cancer, testicular cancer, pancreatic cancer, renal cancer, rectal cancer, thyroid cancer, skin cancer, and head & neck cancer.

4. The method of claim 1, wherein said one statin is chosen among atorvastatin, fluvastatin, pitavastatin, pravastatin, rosuvastatin, simvastatin, cerivastatin, and analogs thereof.

5. The method of claim 1, wherein said EZH2 inhibitor is GSK-126 or tazemetostat.

6. The method of claim 1, wherein said EZH2 inhibitor is GSK-126 or tazemetostat, and wherein said statin is atorvastatin or simvastatin.

7. The method of claim 1, wherein said EZH2 inhibitor is GSK-126 and wherein GSK-126 is administered intravenously to said subject at a dose comprised between 20-1500 mg twice weekly.

8. The method of claim 1, wherein said composition further comprises an effective amount of an anticancer drug.

9. The method of claim 8, wherein said anticancer drug is chosen among platinum compounds, taxane derivatives, topoisomerase 50 inhibitors, hormone therapeutic agents, androgen deprivation agents, androgen receptor agents, serine/threonine kinases inhibitors, tyrosine kinase inhibitors, antiangiogenesis agents, immunotherapeutic preparations, anti-inflammatory drugs, biological preparations having anticancer effects, and anticancer preparations made from natural substances.

10. The method of claim 9, wherein said anticancer drug is cisplatin, carboplatin, and/or oxaliplatin.

11. The method of claim 1, wherein said composition is administered to the subject via intravenous route, parenteral and non-parenteral routes or local injection.

12. The method of claim 1, wherein said composition is administered to relapsed or refractory patients after anticancer standard treatment, radiotherapy or other first or second line of cancer therapies.

13. The method of claim 1, wherein said EZH2 inhibitor is GSK-126 and wherein GSK-126 is administered intravenously to said subject at a dose comprised between 50-1200 mg twice weekly.

14. The method of claim 1, wherein said EZH2 inhibitor is GSK-126 and wherein GSK-126 is administered intravenously to said subject at a dose comprised between 100-1000 mg twice weekly.


SEQUENCE LISTING (18 sequences)

SEQ ID NO: 1 - DNA, 20 bp, synthetic construct
  source 1..20: mol_type = other DNA
gcctgcctttcattggcttc

SEQ ID NO: 2 - DNA, 20 bp, synthetic construct
  source 1..20: mol_type = other DNA
ttccttttctgcgcctcctg

SEQ ID NO: 3 - DNA, 22 bp, synthetic construct
  source 1..22: mol_type = other DNA
actcagttacatagggccatca

SEQ ID NO: 4 - DNA, 20 bp, synthetic construct
  source 1..20: mol_type = other DNA
cgaccgatccttagacacag

SEQ ID NO: 5 - DNA, 20 bp, synthetic construct
  source 1..20: mol_type = other DNA
agctacccctcgagacagat

SEQ ID NO: 6 - DNA, 20 bp, synthetic construct
  source 1..20: mol_type = other DNA
actctccgaagcctgttctg

SEQ ID NO: 7 - DNA, 22 bp, synthetic construct
  source 1..22: mol_type = other DNA
agtgagatctggaggatccaag

SEQ ID NO: 8 - DNA, 20 bp, synthetic construct
  source 1..20: mol_type = other DNA
acaaagacgccatccattcg

SEQ ID NO: 9 - DNA, 20 bp, synthetic construct
  source 1..20: mol_type = other DNA
cttgtgcccgaagcaggaaa

SEQ ID NO: 10 - DNA, 20 bp, synthetic construct
  source 1..20: mol_type = other DNA
ggcatggtgaaacagctgtg

SEQ ID NO: 11 - RNA, 21 nt, synthetic construct
  source 1..21: mol_type = other RNA
  modified_base 10: mod_base = OTHER; note = uracil
  modified_base 12: mod_base = OTHER; note = uracil
  modified_base 14: mod_base = OTHER; note = uracil
  modified_base 17: mod_base = OTHER; note = uracil
gagggaaagtgtatgataatt

SEQ ID NO: 12 - RNA, 21 nt, synthetic construct
  source 1..21: mol_type = other RNA
  modified_base 1: mod_base = OTHER; note = uracil
  modified_base 2: mod_base = OTHER; note = uracil
  modified_base 3: mod_base = OTHER; note = uracil
  modified_base 7: mod_base = OTHER; note = uracil
  modified_base 8: mod_base = OTHER; note = uracil
  modified_base 11: mod_base = OTHER; note = uracil
  modified_base 13: mod_base = OTHER; note = uracil
  modified_base 14: mod_base = OTHER; note = uracil
  modified_base 15: mod_base = OTHER; note = uracil
  modified_base 17: mod_base = OTHER; note = uracil
  modified_base 18: mod_base = OTHER; note = uracil
tttggcttcatctttattgtt

SEQ ID NO: 13 - DNA, 30 bp, synthetic construct
  source 1..30: mol_type = other DNA
gcgcgctagcaccatgggccagactgggaa

SEQ ID NO: 14 - DNA, 30 bp, synthetic construct
  source 1..30: mol_type = other DNA
gcgcacgcgttcaagggatttccatttctc

SEQ ID NO: 15 - DNA, 48 bp, synthetic construct
  source 1..48: mol_type = other DNA
gtttggatttaccgaagcatttgcaaaacgaattttgttacccttgcg

SEQ ID NO: 16 - DNA, 48 bp, synthetic construct
  source 1..48: mol_type = other DNA
cgcaagggtaacaaaattcgttttgcaaatgcttcggtaaatccaaac

SEQ ID NO: 17 - RNA, 33 nt, synthetic construct
  source 1..33: mol_type = other RNA
  modified_base 2: mod_base = OTHER; note = uracil
  modified_base 3: mod_base = OTHER; note = uracil
  modified_base 6: mod_base = OTHER; note = uracil
  modified_base 7: mod_base = OTHER; note = uracil
  modified_base 8: mod_base = OTHER; note = uracil
  modified_base 9: mod_base = OTHER; note = uracil
  modified_base 15: mod_base = OTHER; note = uracil
  modified_base 18: mod_base = OTHER; note = uracil
  modified_base 19: mod_base = OTHER; note = uracil
  modified_base 23: mod_base = OTHER; note = uracil
  modified_base 27: mod_base = OTHER; note = uracil
attcgttttgcaaatcattcggtaaatccaaac

SEQ ID NO: 18 - RNA, 33 nt, synthetic construct
  source 1..33: mol_type = other RNA
  modified_base 2: mod_base = OTHER; note = uracil
  modified_base 3: mod_base = OTHER; note = uracil
  modified_base 6: mod_base = OTHER; note = uracil
  modified_base 7: mod_base = OTHER; note = uracil
  modified_base 8: mod_base = OTHER; note = uracil
  modified_base 9: mod_base = OTHER; note = uracil
  modified_base 15: mod_base = OTHER; note = uracil
  modified_base 18: mod_base = OTHER; note = uracil
  modified_base 19: mod_base = OTHER; note = uracil
  modified_base 23: mod_base = OTHER; note = uracil
  modified_base 27: mod_base = OTHER; note = uracil
attcgttttgcaaatgcttcggtaaatccaaac